(19) United States
(12) Patent Application Publication
(10) Pub. No.: US 2025/0326797 A1
(43) Pub. Date: Oct. 23, 2025

(54) PROTEIN-PROTEIN INTERACTION MODULATORS AND METHODS FOR DESIGN THEREOF

(71) Applicant: RAMOT AT TEL AVIV UNIVERSITY LTD., Tel Aviv (IL)

(72) Inventors: Maayan GAL, Tel Aviv (IL); Jerome TUBIANA, Tel Aviv (IL); Haim WOLFSON, Tel Aviv (IL)

(21) Appl. No.: 19/218,407

(22) Filed: May 26, 2025

Related U.S. Application Data
(63) Continuation of application No. PCT/IL2023/051250, filed on Dec. 6, 2023.

(30) Foreign Application Priority Data
Dec. 9, 2022 (GB) 2218574.8

(51) Int. Cl. C07K 7/08; G16B 15/30; G16B 35/20

(52) U.S. Cl. CPC C07K 7/08; G16B 15/30; G16B 35/20

(57) ABSTRACT

The present invention provides synthetic peptides capable of binding to calcineurin, having a length of about 14-20 amino acids, having at least 1 amino acid difference from any natural peptide sequence. The present invention further provides compositions including such peptides and uses thereof. Further provided are methods and systems for designing such binding peptides.

### CROSS REFERENCE TO RELATED APPLICATIONS

This application is a Bypass Continuation of PCT Patent Application No. PCT/IL2023/051250 having International filing date of Dec. 6, 2023, which claims the benefit of priority of United Kingdom Patent Application No. 2218574.8, filed Dec. 9, 2022, the contents of which are all incorporated herein by reference in their entirety.

### REFERENCE TO AN ELECTRONIC SEQUENCE LISTING

The contents of the electronic sequence listing (RMT-029-PCT.xml; Size: 28,565 bytes; and Date of Creation: Dec. 3, 2023) is herein incorporated by reference in its entirety.

### FIELD OF THE INVENTION

The present disclosure is generally directed to inhibiting protein-protein interactions, and for computerized methods for identifying and designing peptides capable of inhibiting such interactions. Specifically, the invention relates to peptides capable of inhibiting protein-protein interactions involving calcineurin, and methods for their design.

### BACKGROUND OF THE INVENTION

Protein-protein interactions (PPIs) are essential components in all cell signaling pathways. As such, chemical and biological modulators capable of interfering with specific PPI networks are of great importance for fundamental and applied research. However, the design of PPI inhibitors (especially of small molecules) remains a major challenge, mainly due to the physio-chemical properties of protein-protein interfaces. The latter are typically larger, flatter and more flexible than their counterpart enzymatic active sites. These factors limit the inhibitory potential of small molecules and the accuracy of computational molecular docking tools-which heavily rely on shape complementarity.

Peptides, i.e., relatively short amino acid molecules (<50 aa) with no stable fold, are a promising class of PPI perturbators. They are easy to synthesize and can interfere with native PPI by mimicking the binding site of one of the partners. Their potential coverage is high, as it is estimated that up to 40% of human PPIs involve at least one disordered, peptide-like binding region-particularly in cell signaling and regulatory pathways.

The main challenge of peptide discovery lies in the required exhaustive and accurate exploration of the sequence space, as there are 201-peptides of length L. For L>10, this is well beyond the capabilities of experimental investigation and computational approaches based on molecular docking. Nonetheless, a crucial edge of inhibitory peptide discovery is that protein fragments that bind the target protein already exist in nature. This has laid the basis for peptide discovery protocols: starting from a known protein-protein complex structure, an initial peptide sequence is derived from the binding interface of one partner, and its binding affinity is subsequently optimized by in-silico or in-vitro mutagenesis. However, such protocols typically explore only a local neighborhood of the sequence space, and cannot readily screen for additional desirable properties such as high binding specificity, high solubility, or low immunogenicity.

Recent advances in machine learning sequence generative models (SGM) have proven highly successful at: i) learning the biophysical constraints underpinning the functionality of native proteins from raw sequence data, and ii) rapidly exploring the sequence space towards the design of artificial proteins with native-like functionality. However, training accurate SGM necessitates a large and diverse set of evolutionary-related sequences with similar functionality.

Directly transposing this methodology to peptide design is challenging because although additional binding fragments could also a priori be obtained by homology search, the target PPI may only be conserved in a few eukaryotic organisms, and/or may be mediated by highly conserved short linear motifs (SLIMs). Thus, SGM-guided peptide design has been limited to cases where diverse sequence datasets are available, such as for antimicrobial, anticancer or cell-penetrating peptides.

Yet in many PPIs at least one of the partners is highly multivalent, i.e., it interacts with multiple protein interactors, and the corresponding binding regions are highly overlapping. This provides an opportunity to learn from diverse sequence fragments that are evolutionary-unrelated but have similar binding functionality. One important caveat of learning from natural partners is that many interact only transiently with the target, with low binding affinities in the 10²-10³ micromolar range. Therefore, additional in-silico and/or in-vitro screening for filtering high-affinity peptide binders must complement the SGM.

Calcineurin (CaN) is a heterodimeric calcium-dependent protein phosphatase conserved in metazoans, including a catalytic subunit and a regulatory subunit. It activates T cells of the immune system by upregulating expression of interleukin 2, which stimulates growth and differentiation of T cells. Upon calcium chelation and interaction with calmodulin, calcineurin adopts its active conformation in which its catalytic site and binding regions are exposed. Protein substrates binding to calcineurin are characterized by having a conserved PxIxIT consensus sequence and include the family of nuclear factor of activated T-cells (NFAT), conserved in vertebrates. Upon dephosphorylation by calcineurin, NFATs undergo conformational changes that expose nuclear localization motifs, allowing translocation to the nucleus, and in turn, binding to DNA.

Although clinically approved inhibitors of calcineurin exist, including cyclosporine A and tacrolimus, these inhibitors obstruct the calcineurin catalytic site, inhibiting its activity across all substrates and leading to undesirable side effects such as nephrotoxicity and hepatotoxicity.

Accordingly, there is a need in the art for enhanced integrative peptide design methods for the identifying and designing peptide-based modulators, and in particular, peptide-based modulators interfering with the binding of calcineurin to its substrates while keeping its catalytic site available.

### SUMMARY OF INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

In the present invention, the inventors have been able to design new artificial peptides, which were subsequently shown to be capable of binding to calcineurin with a low IC₅₀, by training a model with sequences of fragments from proteins which were known to bind to calcineurin. Such fragments may be used to inhibit calcineurin protein-protein interactions.

Accordingly, in some embodiments, the present invention provides peptides which are capable of inhibiting calcineurin protein-protein interactions.

According to some embodiments, there are provided herein synthetic peptides capable of binding to calcineurin, having a length of about 14-20 amino acids, having at least 1 amino acid difference from any natural peptide sequence, having a sequence conforming to a consensus sequence selected from SEQ ID NO: 18, SEQ ID NO: 19, and SEQ ID NO: 20, and which bind to calcineurin with an IC₅₀ of about 250 μM or less. Further provided are compositions including the same and uses thereof.

In some embodiments, further provided herein are computerized methods and systems for the design of peptides inhibiting protein-protein interactions (PPI). Such improved integrative peptide design methods include, inter alia, the steps of (i) construction of multiple alignments of putatively binding fragments extracted from known and presumed binders; (ii) training and validation of an SGM, and generation of a library of candidate peptide sequences; and (iii) filtering of the library by in-silico flexible protein-peptide docking and optionally in-vitro microarray chip binding assay, to thereby identify potential candidates.

According to some embodiments, there is provided herein a peptide design method (also referred to herein as peptide design protocol), utilizing a machine learning generative model. After identifying putative natural binding fragments by homology search, a compositional generative model suitable for Multiple Sequence Alignments, such as Boltzmann Machine, Restricted Boltzmann Machine or autoregressive models is trained and sampled to yield a large number (hundreds or more) of diverse candidate peptides. The latter candidate peptides are further filtered via flexible molecular docking and optionally in in-vitro microchip-based binding assay.

Thus, the present disclosure relates to a computerized method and system of integrating protein interaction and sequence databases, generative modeling, molecular docking and interaction assays to enable the discovery of novel protein-protein interaction modulators. Specifically, the present disclosure relates to a method for characterizing protein-protein interactions and designing novel protein-protein interaction modulators.

In some embodiments, the synthetic peptide has about 1-6 amino acid differences from a natural peptide sequence that has the highest sequence identity with the synthetic peptide. In some embodiments, the synthetic peptide has a length of about 16 amino acids.

In some embodiments, the peptide sequence is most similar to a natural peptide sequence which is part of a protein selected from TRESK, AKAP79, and RIPOR2. In some embodiments, the peptide sequence comprises a sequence conforming to a consensus sequences as set forth in SEQ ID NO: 18, and is most similar to a natural peptide sequence which is part of the TRESK protein. In some embodiments, the peptide sequence comprises a sequence conforming to a consensus sequences as set forth in SEQ ID NO: 19, and is most similar to a natural peptide sequence which is part of the AKAP79 protein. In some embodiments, the peptide sequence comprises a sequence conforming to a consensus sequences as set forth in SEQ ID NO: 20, and is most similar to a natural peptide sequence which is part of the RIPOR2 protein.

In some embodiments, the synthetic peptide is selected from SEQ ID Nos: 5-10 and 21-28.

In some embodiments, the binding is determined by competition with a PxIxIT motif-containing peptide. In some embodiments, the PxIxIT motif-containing peptide has a sequence according to SEQ ID NO: 4.

In some embodiments, the present invention provides a pharmaceutical composition comprising at least one synthetic peptide as defined herein, and a pharmaceutically acceptable carrier.

In some embodiments, the present invention provides the synthetic peptide disclosed herein or the pharmaceutical composition disclosed herein for use in inhibiting calcineurin activity.

In some embodiments, the present invention provides the synthetic peptide or the pharmaceutical composition disclosed herein for use in peptide-based therapy for treating an autoimmune disease or an inflammatory disease, or for preventing graft rejection following transplantation.

In some embodiments, the present invention provides a method of treating a subject in need of immunosuppression, comprising administering to the subject a therapeutically effective dose of the synthetic peptide or the pharmaceutical composition disclosed herein.

In some embodiments, the subject suffers from an autoimmune or an inflammatory disease or condition, or is a post-transplantation patient.

In some embodiments, the present invention provides a kit comprising at least one synthetic peptide disclosed herein, and instructions for use.

In some embodiments, the present invention provides a method for designing protein-protein interaction modulator peptides, the method comprising the steps of:
identifying a binding region of a target protein;
identifying at least one substrate having a peptide-like binding fragment which interacts with the binding region of the target protein;
performing a homology/orthology search across sequence databases to identify additional homologous peptide-like binding fragments;
creating a data set comprising at least one peptide-like binding fragment and at least one homologous peptide-like binding fragment;
training a sequence generative model (GSM) to generate a library of candidate peptide sequences; and
screening the library of candidate peptide sequences for peptides capable of binding to the binding region of the target protein.

In some embodiments, the screening comprises in-silico screening and/or in-vitro screening.

In some embodiments, the in-silico screening comprises estimating the binding strength of at least one candidate peptide to the target protein by a protein-peptide docking algorithm.

In some embodiments, the -silico screening comprises applying a template-based docking with Modeller followed by flexible backbone refinement with PepCrawler, or applying ab initio docking with AlphaFold-Multimer followed by ProteinMPNN for scoring.

In some embodiments, the in-vitro screening comprises a qualitative binding assay to evaluate direct binding of at least one candidate peptide to the target protein.

In some embodiments, the qualitative binding assay comprises a peptide microarray.

In some embodiments, the method further comprises the step of:
performing a quantitative binding assay on at least one candidate peptide to determine the ability of the at least one candidate peptide to compete with the binding of the at least one substrate.

In some embodiments, the sequence generative model comprises a Boltzmann Machine and/or autoregressive model.

In some embodiments, the Boltzmann Machine comprises a compositional Restricted Boltzmann Machine.

In some embodiments, a two-stage sequence-based statistical filtering protocol is applied to results of the homology/orthology search to eliminate presumed non-interacting homologs.

In some embodiments, the present application provides a system for designing protein-protein interaction modulator peptides, the system comprises a processing circuitry; and a memory, the memory containing instructions that, when executed by the processing circuitry, configure the system to execute the method disclosed herein.

In some embodiments, the present application provides a non-transitory computer readable medium having stored thereon instructions for causing a processing circuitry to execute the method for the design of peptide inhibitors of a target PPI disclosed herein.

In addition to the exemplary aspects and embodiments described above, further aspects and embodiments will become apparent by reference to the figures and by study of the following detailed descriptions.

### BRIEF DESCRIPTION OF DRAWINGS

The patent or application file contains at least one drawing executed in color. Copies of this patent or patent application publication with color drawings will be provided by the Office upon request and payment of necessary fec.

The invention will now be described in relation to certain examples and embodiments with reference to the following illustrative figures.

FIGS. 1A-B. Overview of the Calcineurin-NFAT complex. FIG. 1A—3D image of Structure of Calcineurin bound to representative SLIM-containing peptides (pdb codes: 5sve, 2p6b). Calcineurin is shown in molecular surface representation, and is colored by propensity to bind disordered regions (from white=low to dark red=high), based on ScanNet. The catalytic site is colored in blue (marked by arrow 2). Both the catalytic and the regulatory (circled in green (marked by arrow 4) subunits are shown. PxIxIT and LxVP-containing peptides are shown in stick representation (in magenta (arrow 6) and yellow (arrow 8), respectively). FIG. 1B shows a table summarizing Sequence alignment of the PxIxIT short linear motifs that bind calcineurin;

FIG. 2. A schematic illustration of overview of computerized method for design of peptide inhibitors of a target PPI, according to some embodiments;

FIGS. 3A-3H—. Overview of Generative modeling of PxIxIT binding motifs, according to some embodiments. FIG. 3A—Schematic view of the generative approach. A “smooth” probability distribution over the whole sequence space is learnt from a limited number of samples. Unseen sequences with high probability are potential novel binders, whereas regions with low probability are likely non-functional proteins. FIG. 3B—Depiction of exemplary Restricted Boltzmann Machine (the parametric form chosen). FIGS. 3C-3D CRBM-predicted mutational landscapes for NFATc2 and AKAP79 peptides, respectively. Red, white and blue entries correspond respectively to beneficial, neutral and deleterious mutations. FIG. 3E—Comparison between cRBM-predicted mutational landscapes and deep mutational scans (DMS) of change in binding affinity. Four DMS were performed taking as wild type the PVIVIT, PKIVIT, NFATc2 and AKAP79 peptides. Spearman correlation coefficients are annotated. FIGS. 3F, 3G and 3H show selected examples of sequence motifs learnt by the cRBM (FIG. 3F), together with their activity distribution (FIG. 3G-3H) and top-activating sequences. Motif 1 is gene-specific, whereas motifs 2 and 3 are shared by multiple genes.

FIGS. 4A-F—. Schematic overview of the medium-throughput filtering by structural modeling and microarray screening. FIG. 4A—Depiction of the structural modeling method: after alignment to the known PxIxIT binding site, an efficient flexible backbone structure refinement algorithm is applied to estimate the docking energy. FIG. 4B Histogram of docking energy scores for the generated peptides and selected controls (lower is better; normalized to zero mean and unit variance). FIG. 4C Coefficients of the equivalent single-site model fitted by sparse linear regression, shown in weight logo representation. At each position, the height of the letter is proportional to the corresponding coefficient of the regression; residues with large negative coefficients (e.g. hydrophobic residues at the motif locations) contribute favorably to the docking score. Colors indicate physical property (black=hydrophobic, red=negatively charged, etc.). FIG. 4D—Per-gene distribution of docking scores across natural fragments (lower is better). The docking protocol qualitatively discriminates between obligate and transient interactions. FIG. 4E Overview of the microarray screening. Peptides are printed on the chip (two circles per peptide). After pouring of CaN and subsequent washing, fluorescent-tagged, a CaN-targeting antibody is overlaid and an image is taken. Fluorescent spots indicate strong CaN binders. FIG. 4F Scatter plot of the sequence likelihood (normalized by length, higher is better) against fluorescence level (higher is better).

FIG. 5. FP competition assay of selected peptides for the binding of CaN to PVIVIT peptide. Peptides shown: rbmAKAP79 (blue full circle), rbmRIPOR2_2 (blue square); rbmTRESK (blue diamond); AKAP79 (natural, upside down red triangle); PVIVIT (control, black square); C16Orf74 (natural, right-side up red triangle); TRESK (natural, empty red circle).

FIGS. 6A-C. Construction and refinement of the multiple fragment alignment (MFA). FIG. 6A presents an overview of the method for constructing the multiple fragment alignment. FIGS. 6B-C cRBM-based refinement of the MFA obtained from the homology search: A cRBM model is trained on the MFA and likelihood scores are computed for all sequences (higher is better). Sequences with low likelihood values do not share the main conservation and coevolution patterns of the others and may be discarded. To determine a cut-off, sequences are grouped by likelihood, and the sequence profile of each subgroup is visualized (FIG. 6C). Sequences with Z<−0.3 do not feature any conservation pattern, and are considered outliers.

FIG. 7A-C. Selected data visualization of the multiple fragment alignment. FIGS. 7A-B T-SNE visualization of the MFA, colored by phyla/gene reveals that fragments mainly cluster by gene. (FIG. 7C Gene-specific sequence profiles, revealing a diversity of conserved binding motifs. “B” denotes the number of unique fragments found.

FIGS. 8A-E. An overview of the sequence model. FIGS. 8A-B—Model selection protocol. A grid search is performed over the number of hidden units and values of sparse penalty. The model that achieves the best compromise between accuracy (high likelihood) and interpretability (low motif sparsity) is selected (black triangle). FIG. 8C—Distribution of likelihood changes upon mutation grouped by position. The distribution is computed over all 19 possible mutations at each position for 100 representative fragments from the alignment (randomly selected by Kmeans++ algorithm). Positions with low average values are less tolerant to mutations and presumably more important for functionality. FIG. 8D—Effective epistatic couplings learnt by the model, indicating significant covariation between core and flanking residues. FIG. 8E—The quality-diversity trade-off of generated sequences. Scatter plot of sequence likelihood against number of mutations to the closest natural fragment. Sequences with likelihood similar or higher to the one of natural sequences but distinct from them can be generated. Conversely, random or PSSM-generated sequences are further away but distinguishable from natural fragments.

FIGS. 9A-E. Microassay experiment data processing. FIGS. 9A and 9B—Scatter plot of the fluorescence intensity level against the row and column index along the chip for one of the seven repeats (one point per peptide). A trend is fitted (red curve) and removed before further analysis. FIG. 9C Distribution of fluorescence intensity level (logarithmic scale, normalized to zero mean and unit variance, averaged over the seven repeats), for peptides printed at the border and at the interior of the chip. Spillover from the neighboring fluorescent tags results in higher fluorescence levels for border peptides; positive hits along the border were ignored in downstream analysis. FIG. 9D Scatter plot of the docking energy score against the fluorescence level. FIG. 9E Single-site model fit of the docking energy scores by LASSO regression: scatter plot between docking energies and cross-validated predictions.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

It is estimated that over half a million protein-protein interactions (PPIs) occur in the cell, among which many play important physiological roles and are potential therapeutic targets. However, discovery of PPI modulators, especially in the form of small organic molecules, is hampered by the inherent physio-chemical properties of PPI interfaces, limited availability of structural data and accuracy of docking tools. This often prompted the conclusion that PPIs are “undruggable” targets. Design of peptides capable of binding a target protein with high affinity and specificity, and interfering with its native protein-protein interactions, may provide reagents for peptide-based therapy, as well as for basic systems biology research, structural characterization of protein-protein interactions, and drug discovery campaigns. However, rational peptide design remains a major challenge, owing to the large search space, difficulty to estimate at high throughput the binding affinity and specificity in vitro or in silico, and necessity to integrate multiple design constraints.

In some embodiments, the present invention provides a novel integrative method and system for designing peptides targeting a specific binding site of a protein, based on protein fragments extracted from native interaction partners. After identifying putative natural binding fragments by literature and/or homology search, a generative model suitable for multiple sequence alignments (MSA), such as compositional Restricted Boltzmann Machine (cRBM), or autoregressive models is trained and sampled to yield hundreds of diverse candidate peptides. The latter are further filtered via flexible molecular docking and an in-vitro microchip-based binding assay.

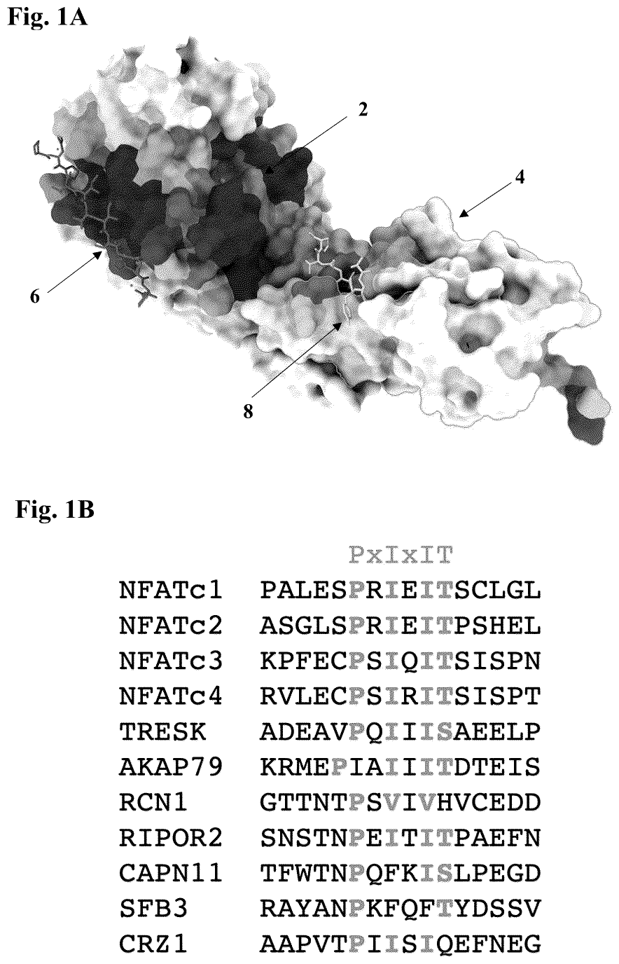

As exemplified herein, the protocol was validated and tested on peptides binding to calcineurin (CaN), a calcium-dependent protein phosphatase involved in various cellular pathways in health and disease. Calcineurin (CaN) is a heterodimeric calcium-dependent phosphatase conserved in metazoans, constituted by a catalytic (˜510 amino acids) and a regulatory subunit (˜170 amino acids), having a structure as shown in FIG. 1A. Upon calcium chelation and interaction with calmodulin (both mediated by the regulatory subunit), CaN adopts its active conformation in which its catalytic site and binding regions are exposed. In turn, CaN substrates—most of which are intrinsically disordered-bind it, enabling dephosphorylation of serine and threonine residues by CaN. The NFAT family—a set of five transcription factors conserved in vertebrates—are known examples of substrate of CaN. Upon dephosphorylation by CaN, they undergo conformational changes that expose nucleus localization motifs, allowing translocation to the nucleus, and in turn, binding to DNA. More generally, the CaN signaling network was systematically investigated in mammals and yeast using combinations of in-vivo, in-vitro and in-silico methods, and at least 29 and 38 protein substrates were identified with high confidence respectively for human and yeast.

To determine the regions tethering the substrates, the ScanNet web server was used to predict binding sites of intrinsically disordered proteins (shown as red scale coloring in FIG. 1A). In addition to the catalytic site, two substrate binding sites are found. Previous studies showed that they recognize two SLIMs: PxIxIT and Lx VP, where uppercase letters stand for conserved residues and x represents alternate amino acids. Both motifs: i) bind Cn in isolation (crystal structures of representative Cn-bound PxIxIT and Lx VP motifs are depicted in respectively magenta and yellow of FIG. 1A); and ii) are conserved across a wide range of substrates as shown for the NFAT isoforms in FIG. 1B.

According to some embodiments, as exemplified herein, the method was applied to the CaN-PxIxIT complex. In a single screening round, multiple 16-length peptides with up to six mutations from their closest natural sequence were identified, where 7/10 designe¾eptides and ¾ natural peptides successfully interfered with the binding of calcineurin to its substrates.

The most successful of these peptides were a previously overlooked natural peptide featuring a C-terminal proline-rich motif (derived from C16Orf74, SEQ ID NO: 1), and a designed recombinant peptide harboring six mutations from its closest natural counterpart (rbmTRESK, similar to a peptide derived from the TRESK protein, SEQ ID NO: 5).

A general consensus sequence was calculated based on the binding peptides found and by taking into account permissible changes which were predicted not to affect the binding to CaN. The general consensus sequence is [A/T/S]X[P/V][E/K/Q/R/S/G]I[T/V/I][I/V][D/H/Q/S/T]XXE.

Accordingly, in some embodiments, the present invention provides a peptide capable of binding to calcineurin, and having a consensus sequence defined by [A/T/S]X[P/V][E/K/Q/R/S/G]I[T/V/I][I/V][D/H/Q/S/T]XXE, wherein “X” may be any amino acid.

Another way to present the consensus sequence is: X₁X₂X₃X₄IX₆X₇X₈X₉X₁₀E, wherein X₁=A, T, or S; X₂ may be any amino acid; X₃=P or V; X₄=E, K, Q, R, S, or G; X₆=T, V, or I; X₇=I or V; X₈=D, H, Q, S, or T; and X₉ and X₁₀ may be any amino acid.

In some embodiments, the peptide is a synthetic peptide. In some embodiments, the synthetic peptide is a non-natural peptide, having at least one amino acid difference from a sequence of any natural peptide. In some embodiments, the synthetic peptide has at least about 1-6 amino acids different from any natural peptide sequence. In some embodiments, the synthetic peptide has at least about 1, 2, 3, 4, 5, or 6 amino acids different from any natural peptide sequence. In some embodiments, the synthetic peptide has about 1-6 amino acids different from a natural peptide sequence that has the highest sequence identity with the synthetic peptide. In some embodiments, the synthetic peptide has at least about 1, 2, 3, 4, 5, or 6 amino acids different from a natural peptide sequence that has the highest sequence identity with the synthetic peptide.

The term “synthetic peptide”, as used herein, relates to a molecule comprised of a relatively short sequence of amino acids (usually less than 50) that is artificially synthesized. The synthesis may be performed by any acceptable process for peptide synthesis, such as in-solution or solid phase chemical synthesis methods. In some embodiments, the synthetic peptide has a non-natural sequence, i.e., a sequence not found in nature.

The term “natural peptide”, as used herein, relates to a peptide which appears in nature, and may be part of a natural protein. The length of the natural peptide is not important and it is assumed that the identity between the synthetic peptide and the natural peptide is determined based on the best alignment, generally minimizing differences and gaps between the two sequences, such as a BLAST (Basic Local Alignment Search Tool, from the National Center for Biotechnology Information, NCBI) alignment or similar. In some embodiments, the natural peptide is of about the same length as the synthetic peptide, such as up to about 5 amino acids longer or shorter.

In some embodiments, binding to calcineurin is determined by competition with a PxIxIT-motif-containing peptide. The competition may be conducted by a suitable test, such as a fluorescence polarization (FP) competition assay, an enzyme-linked immunosorbent assay (ELISA), or a microscale thermophoresis assay. In some embodiments, the PxIxIT motif-containing peptide has a sequence according to SEQ ID NO: 4.

In some embodiments, the synthetic peptide binds calcineurin with an IC₅₀ of about 250 μM or less. In some embodiments, the synthetic peptide binds calcineurin with an IC₅₀ of about 0.1-250, or 1-200 μM. In some embodiments, the synthetic peptide binds calcineurin with an IC₅₀ of about 200, 150, 100, 50 μM or less. IC₅₀ quantitates the binding affinity of the peptide to calcineurin.

In some embodiments, the synthetic peptide has a length of about 8-20 amino acids (aa). In some embodiments, the synthetic peptide has a length of about 10-20, 14-20, 14-18, or about 16 aa.

In some embodiments, the present application provides a synthetic peptide capable of competing with a PxIxIT motif-containing peptide on binding to calcineurin, wherein the synthetic peptide has a length of about 14-20 amino acids, has at least 1 amino acid difference from any natural peptide sequence, includes a sequence conforming to a consensus sequence defined by [A/T/S]X[P/V][E/K/Q/R/S/G]I[T/V/I][I/V][D/H/Q/S/T]XXE, wherein “X” may be any amino acid, and binds calcineurin is with an IC₅₀ of about 250 μM or less.

Since the computational methods used for designing the sequences included models trained with natural sequences expected to bind to calcineurin, a peptide of the invention can be said to be derived from a protein which includes a sequence that is most similar to the synthetic peptide.

The term “derived from”, as used herein with reference to a synthetic peptide being derived from a protein, relates to a protein having a sequence which is the most similar to the synthetic peptide. In other words, the synthetic peptide is said to be derived from a protein which has a sequence which is closest to the synthetic peptide. The closest protein sequence may be determined by any suitable method, for example, by a sequence alignment software, such as BLAST.

Accordingly, in some embodiments, the synthetic peptide is derived from a calcineurin binding protein.

In some embodiments, the synthetic peptide is derived from a protein selected from TRESK, AKAP79, and RIPOR2. In some embodiments, the synthetic peptide is derived from TRESK. In some embodiments, the synthetic peptide is derived from AKAP79.

In some embodiments, the consensus sequence is selected from SEQ ID NO: 18, SEQ ID NO: 19, and SEQ ID NO: 20.

In some embodiments, the consensus sequence is a TRESK consensus sequence defined as AXP[E/K/Q/R/S]I[T/V/I][I/V][D/H/Q/S/T]XXE (SEQ ID NO: 18).

In some embodiments, the consensus sequence is an AKAP79 consensus sequence defined as [A/T]GVGIVIT[I/P/V]TE (SEQ ID NO: 19).

In some embodiments, the consensus sequence is a RIPOR2 consensus sequence defined as [A/S]NPEIT[I/V]TXAE (SEQ ID NO: 20).

The SEQ ID NO: 18 consensus may also be presented as AX₂PX₄IX₆X₇X₈X₉X₁₀E, wherein X₂ may be any amino acid; X₄=E, K, Q, R, or S; X₆=T, V, or I; X₇=I or V; X₈=D, H, Q, S, or T; and X₉ and X₁₀ may be any amino acid.

The SEQ ID NO: 19 consensus may also be presented as X₁GVGIVITX₉TE, wherein X₁=A or T; and X₉=I, P, or V.

The SEQ ID NO: 20 consensus may also be presented as X₁NPEITX₇TX₉AE, wherein X₁=A or S; X₇=I or V; and X₉ may be any amino acid.

In some embodiments, the synthetic peptide is derived from the TRESK protein and comprises a sequence conforming to a consensus sequences as set forth in SEQ ID NO: 18.

In some embodiments, the synthetic peptide is derived from the AKAP79 protein and comprises a sequence conforming to a consensus sequences as set forth in SEQ ID NO: 19.

In some embodiments, the synthetic peptide is derived from the RIPOR2 protein and comprises a sequence conforming to a consensus sequences as set forth in SEQ ID NO: 20.

Several peptides, resulting from the model of the invention and listed in Table 2, were tested and showed capability of competing on binding to CaN in an FP competition assay with a PVIVIT peptide (SEQ ID NO: 4).

In some embodiments, the sequence of the synthetic peptide includes a sequence selected from SEQ ID NOs: 5-10 and 21-28. In some embodiments, the sequence of the synthetic peptide includes a sequence selected from SEQ ID NOs: 5-10.

In some embodiments, the sequence of the synthetic peptide includes a TRESK-derived sequence selected from SEQ ID NOs: 5, 7 and 21-23, or form SEQ ID NOs: 5 and 7.

In some embodiments, the sequence of the synthetic peptide includes a AKAP79-derived sequence selected from SEQ ID NOs: 6, 8, 24, and 25, or SEQ ID NOs: 6 and 8.

In some embodiments, the sequence of the synthetic peptide includes a RIPOR2-derived sequence selected from SEQ ID NOs: 9, 10, and 26-28, or SEQ ID NOs: 9 and 10.

In some embodiments, the present invention provides a pharmaceutical composition including at least one synthetic peptide as disclosed herein, and a pharmaceutically acceptable carrier.

In general, definitions and embodiments mentioned above and which may be relevant to the pharmaceutical composition also apply here, and vice versa. Some particularly relevant embodiments may be pointed out or explicitly repeated.

Pharmaceutical compositions for use in accordance with the present invention may be formulated in any conventional manner using one or more physiologically or pharmaceutically acceptable carriers or excipients. The carrier(s) must be “acceptable” in the sense of being compatible with the other ingredients of the composition, not being deleterious to the recipient thereof, and not significantly interfering with the activity of the compound of the invention, or of any other active ingredient in the pharmaceutical composition.

The term “carrier” refers to a diluent, adjuvant, excipient, or vehicle with which the active agent is administered.

Designing high-affinity peptides can i) provide structural insights into transient PPIs that are challenging to characterize experimentally, ii) suggest pharmacophore hypotheses for in-silico screening of small molecules iii) facilitate small molecule screening based on in-vitro competition assay (e.g. by Fluorescence Polarization) and iv) lead to peptidomimetics-based therapeutics.

In some embodiments, the present invention provides the synthetic peptides of the invention or the pharmaceutical composition of the invention for use for use in inhibiting calcineurin activity.

In some embodiments, the inhibiting is conducted in vitro or ex vivo, such as on a sample of cells taken from a patient. In some embodiments the inhibiting is conducted in vivo.

In some embodiments, the present invention provides the synthetic peptides of the invention or the pharmaceutical composition of the invention for use for use in peptide-based therapy for inhibiting calcineurin activity.

In general, definitions and embodiments mentioned above and which may be relevant to the use embodiments also apply here, and vice versa. Some particularly relevant embodiments may be pointed out or explicitly repeated.

Calcineurin is involved in the production of interleukin-2, which promotes the development and proliferation of T cells, as part of the adaptive immune response. Accordingly, inhibition of calcineurin activity cases immunosuppression.

Relevant diseases or conditions that may be treated by the peptides of the invention include diseases or conditions treatable by calcineurin inhibitors, or by immunosuppressive agents, such as cyclosporin, voclosporin, pimecrolimus, and tacrolimus. Such conditions include autoimmune diseases and inflammatory diseases. Additionally, immunosuppression is required in post-transplantation patients, for preventing grant rejection.

Accordingly, in some embodiments, the present invention provides the synthetic peptide or the pharmaceutical composition of the invention for use in peptide-based therapy for treating an autoimmune disease or an inflammatory disease, or for preventing graft rejection following transplantation.

In some embodiments, the present invention provides a method of treating a subject in need of immunosuppression, including administering to the subject a therapeutically effective dose of at least one synthetic peptide of the invention or the pharmaceutical composition of the invention.

In general, definitions and embodiments mentioned above and which may be relevant to the method of treatment embodiments also apply here, and vice versa. Some particularly relevant embodiments may be pointed out or explicitly repeated.

In some embodiments, the subject suffers from an autoimmune or an inflammatory disease or condition, or is a post-transplantation patient. Non-limiting examples for autoimmune or inflammatory diseases or conditions include lupus nephritis, idiopathic inflammatory myositis, interstitial lung disease, and atopic dermatitis.

The term “post-transplantation patient” relates to a subject who has gone through organ transplantation, and is in need of receiving immunosuppression for preventing the development of graft rejection.

The term “treating” or “treatment”, as used herein, refers to means of obtaining a desired physiological effect. The effect may be therapeutic in terms of partially or completely curing a disease and/or symptoms attributed to the disease. The term includes inhibiting the disease, i.e. arresting its development; or ameliorating the disease, i.e. causing regression of the disease, e.g., by eliminating or ameliorating its symptoms.

The term “preventing”, as used herein, refers to causing a condition or symptoms thereof not to appear in the subject, or delaying the onset of such condition or symptoms, such that they do not appear at the time they are expected to appear based on similar cases, or causing the condition or symptoms to appear at a diminished level.

As used herein, the terms, “subject” or “individual” or “animal” or “patient” or “mammal,” refers to any subject, particularly a mammalian subject, for whom diagnosis, prognosis, or therapy is desired, for example, a human.

The term “therapeutically effective amount” as used herein means an amount of the peptide that will elicit the biological or medical response of a tissue, system, animal or human that is being sought, i.e. immunosuppression. The amount must be effective to achieve the desired therapeutic effect as described above, depending inter alia on the type and severity of the condition to be treated and the treatment regime. The therapeutically effective amount is typically determined in appropriately designed clinical trials (dose range studies) and the person skilled in the art will know how to properly conduct such trials to determine the effective amount.

Methods of administration may include parenteral, e.g., intravenous, intraperitoneal, intramuscular, subcutaneous; mucosal (e.g., oral, sublingual, intranasal, buccal, vaginal, rectal, intraocular), intrathecal, topical, and intradermal routes. Administration can be systemic or local. In certain embodiments, the pharmaceutical composition is adapted for parenteral administration. In some embodiments, the administration is by injection.

In some embodiments, the present invention provides a kit including at least one synthetic peptide as disclosed herein, and instructions for use.

In some embodiments, the present invention provides the kit of the invention for use in peptide-based therapy for inhibiting calcineurin activity.

In general, definitions and embodiments mentioned above and which may be relevant to the kits embodiments also apply here, and vice versa. Some particularly relevant embodiments may be pointed out or explicitly repeated.

According to some embodiments, there are provided herein methods (protocols) and systems for the identification and characterization of protein-protein interactions (PPI) and for the design of peptides inhibiting the protein-protein interactions.

According to some embodiments, the herein disclosed peptide design protocol was exemplary implemented and evaluated on the PPI between Calcineurin (Cn), a calcium-dependent protein phosphatase, and its substrates containing the conserved SLIM PxIxIT. However, the methods can be applied to any type of suitable target protein. Thus, although the protocol disclosed herein was exemplified with the highly multivalent and thoroughly-studied Cn, it is contemplated that other protein targets of interest enjoy similar feats and, therefore, it is also envisioned that this protocol is also applicable towards the discovery of other PPI modifiers as well.

According to some embodiments, there is provided an integrative approach to design peptides targeting a specific binding site of a target protein, based on protein fragments extracted from native interaction partners. After identification of native partners together with their interacting fragments using, for example, available experimental data and homology search, a sequence generative model may be trained and sampled from, yielding an in-silico library of a large number (for example, 10³⁻⁴) of “reversed-engineered” peptides. Identified peptides may be subsequently filtered by a cost-effective and medium-throughput approach (template-based docking and microarray binding assay). Finally, a focused list selected peptides may be prioritized and their ability to interfere with the target protein-protein interaction(s) may then be quantified by suitable assays.

According to some embodiments the robustness of the SGM tools with respect to corrupt training data and the complementarity between evolutionary-based and docking-based approaches synergistically enhance identification of novel peptide binders having improved properties. Without wishing to be bound to any theory or mechanism, while evolutionary-based models alone could not discriminate transient from tight natural binders, structure-based docking alone could not explain amino acid preferences for flanking residues and favored promiscuous, hydrophobic side-chains for central residues.

According to some embodiments, the methods include one or more of the general steps of:
identifying a binding region of a target protein;
identifying at least one substrate having a peptide-like binding fragment which is capable of interacting with the binding region of the target protein;
performing a homology/orthology search across sequence databases to identify additional homologous peptide-like binding fragments;
creating a data set including at least one peptide-like binding fragment and at least one homologous peptide-like binding fragment;
training a sequence generation model to generate a library of candidate peptide sequences; and
screening the library of candidate peptide sequences for candidate peptides configured to bind to the binding region of the target protein.

Thus, by the advantageous methods disclosed herein, novel PPI inhibiting peptides may be designed. Such inhibitory peptides may exhibit one or more enhanced properties, such as, increased binding affinity, stability (such as thermal and/or chemical stability); reduced toxicity, and the like, or any combinations thereof.

According to some embodiments, the protein substrates of the target protein (for example, an enzyme) are first identified, for example based on previous experiments together with their binding fragment. Additional interacting orthologs may be identified by homology search, and the corresponding binding regions may be extracted and aligned. An SGM sequence generative model is trained to generate a library of candidate peptides. The latter are screened for affinity by structural modeling and high-throughput binding assay. The best candidates are selected for further low-throughput experimental characterization.

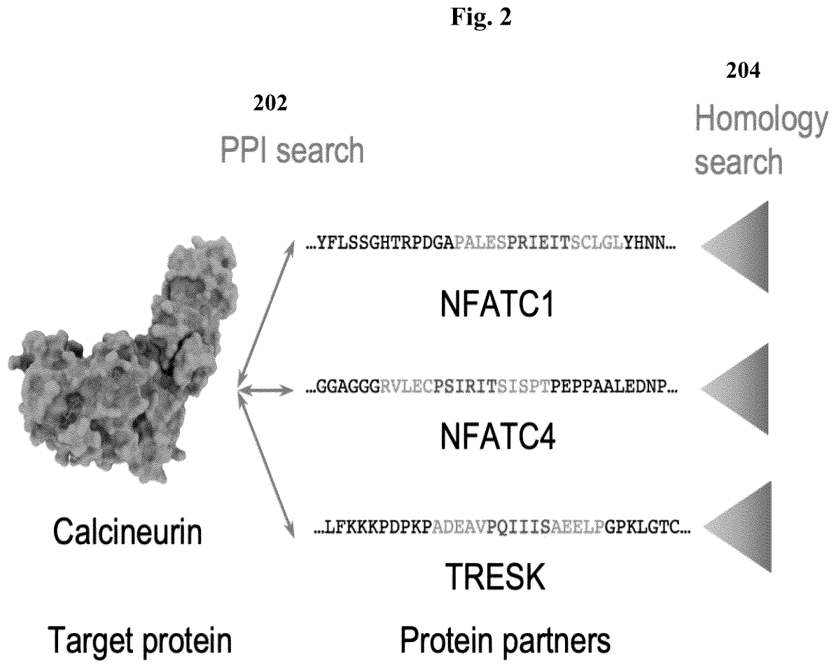

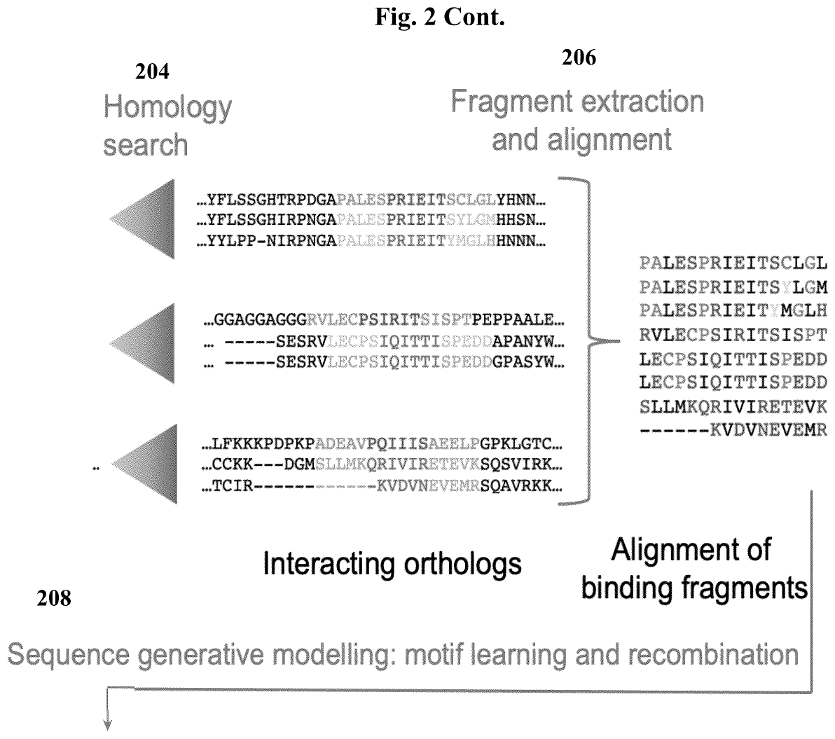

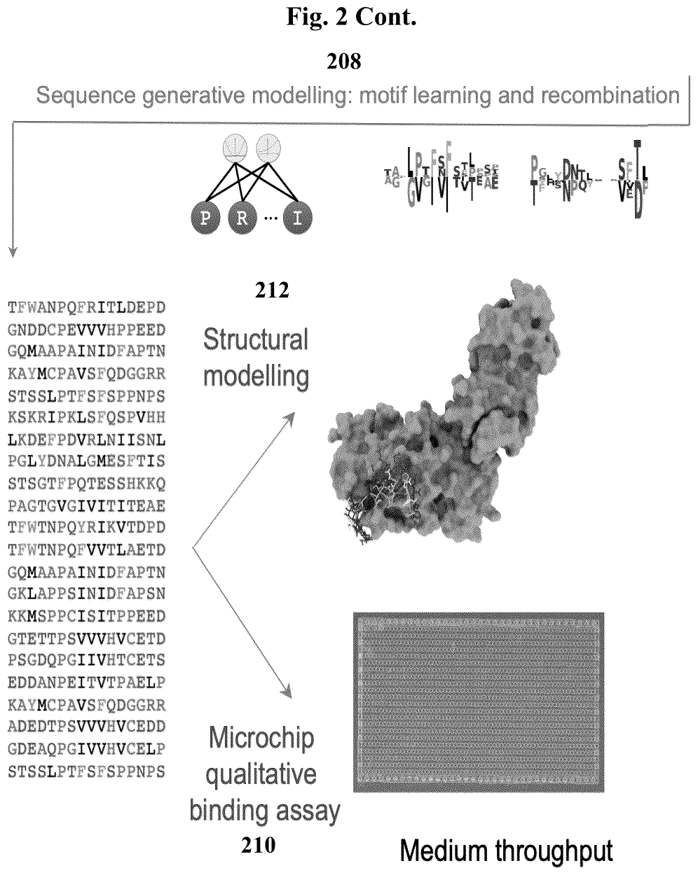

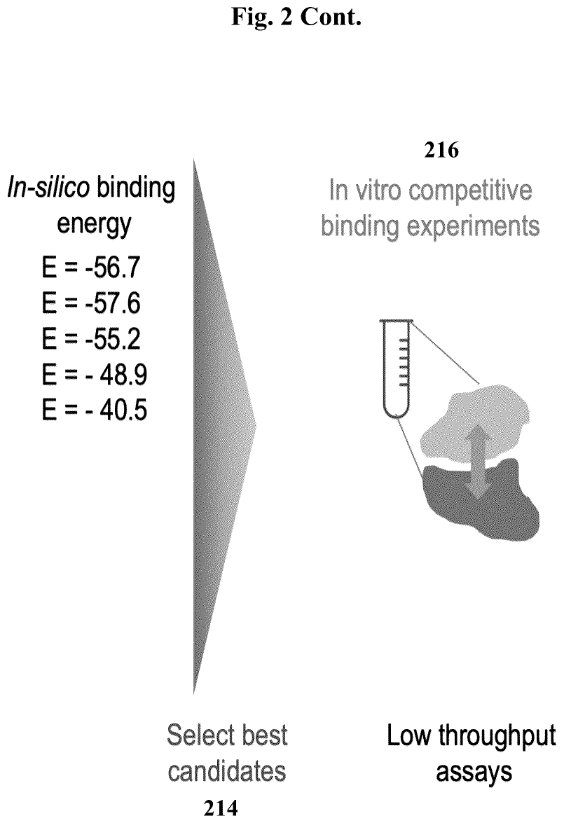

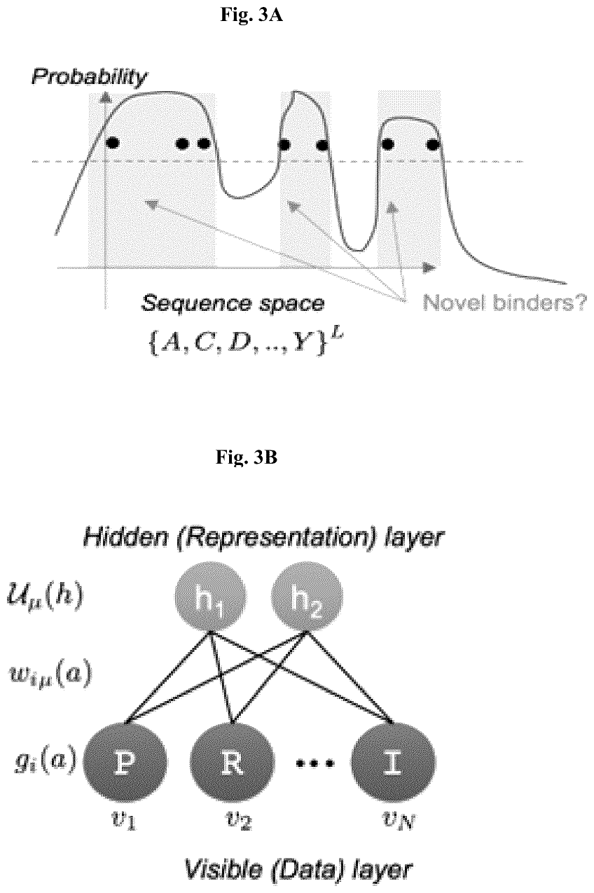

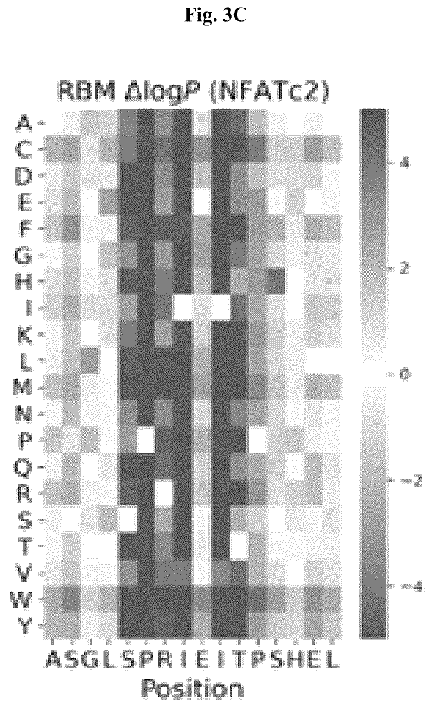

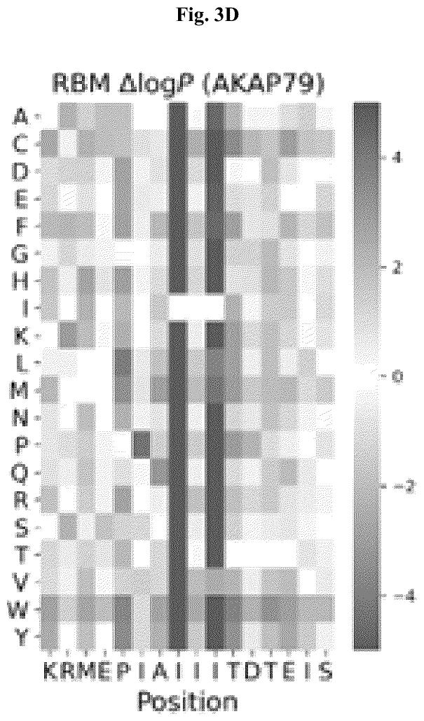

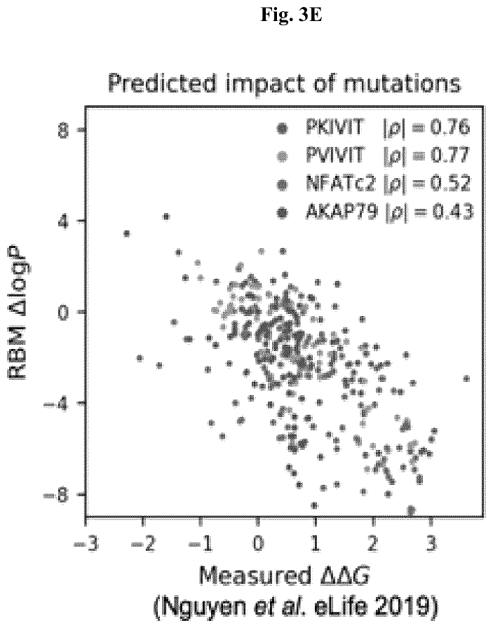

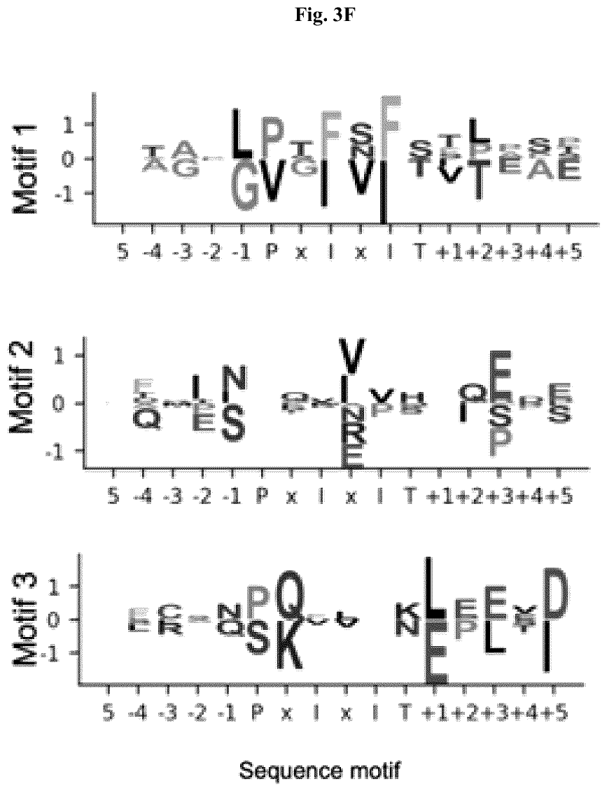

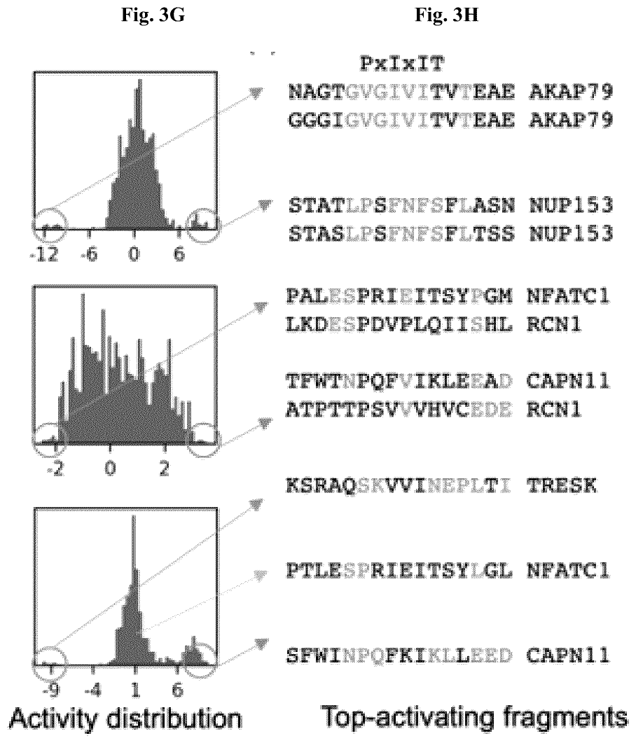

Reference is now made to FIG. 2, which shows steps in a method for the design of peptide inhibitors of a target PPI. For simplicity of explanation, the target protein shown in FIG. 2 is Cn, however, as detailed above, the method may be applied to any suitable target protein. As shown in FIG. 2, at step 202, known Cn-binding fragments are curated, for example, by performing a search on sequence databases (for example, from literature survey). In the example shown in FIG. 2, a list of 67 protein substrates of Cn from human and yeast that have been previously characterized (44,45), together with their corresponding PxIxIT-containing fragment(s). Next, at step 204, data augmentation by homology search is performed. In order to increase the number of potential sequences, and since the obtained number is too limited for meaningful SGM, the set may be first enriched by performing a homology search across sequence databases, to identify additional PxIxIT-like fragments in homologous sequences for each of the listed substrates. Such orthologs may be collected from the Homologene database if available, or via a BLAST search over, for example, UniProt. Importantly, the PPI is not guaranteed to be conserved across all orthologs/paralogs (especially in cases like the Cn signaling networks, which undergo rapid rewiring throughout evolution). Therefore, a two-stage sequence-based statistical filtering protocol may be applied to eliminate presumed non-interacting homologs, by applying step 206. After realignment and deduplication, a multiple sequence alignment of natural, putatively target protein (Cn in this example)-binding fragments is obtained. Next, at step 208, a sequence generative model (SGM) is applied. To this aim, a Boltzmann Machine or other autoregressive model may be trained. In some embodiments, the Boltzmann Machine is a compositional Restricted Boltzmann Machine (cRBM). After quantitatively and qualitatively validating the model learned (Sec, for example FIGS. 3A-G, detailed hereinbelow), it may be used to generate a diverse library of a large number (for example, 10²⁻³) candidate peptide sequences.

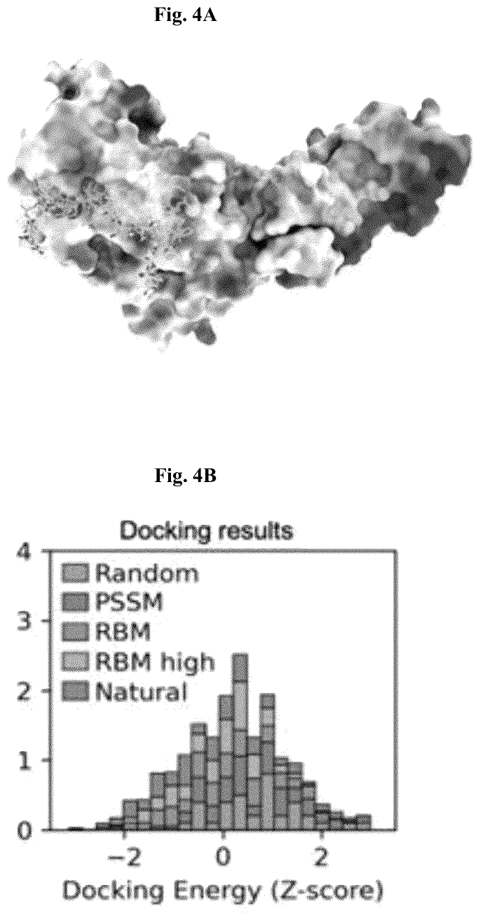

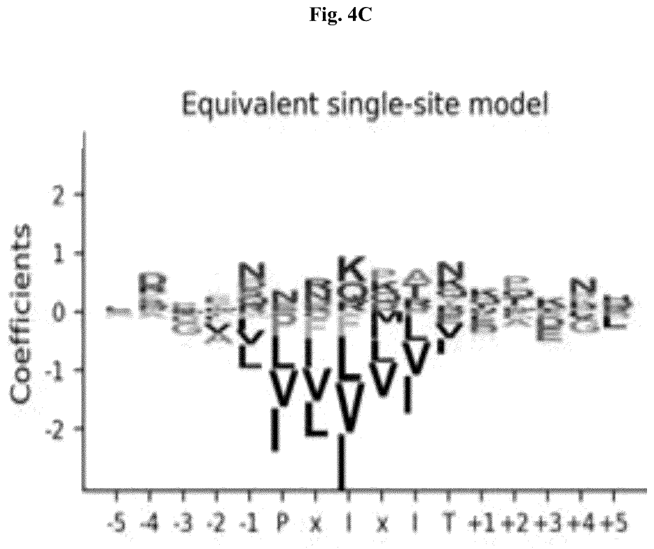

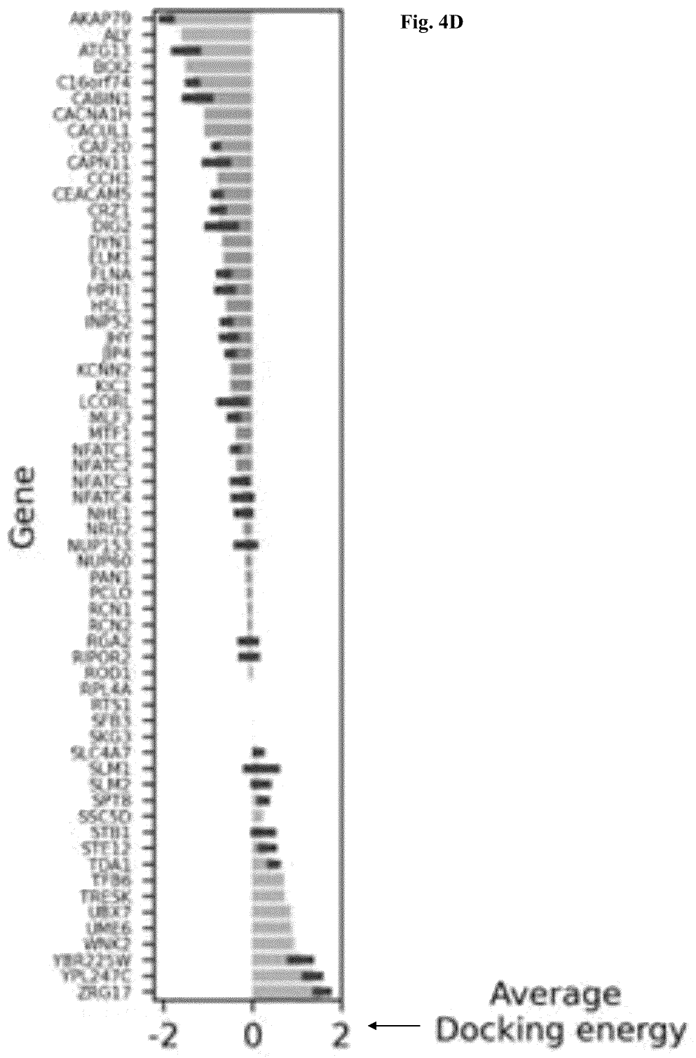

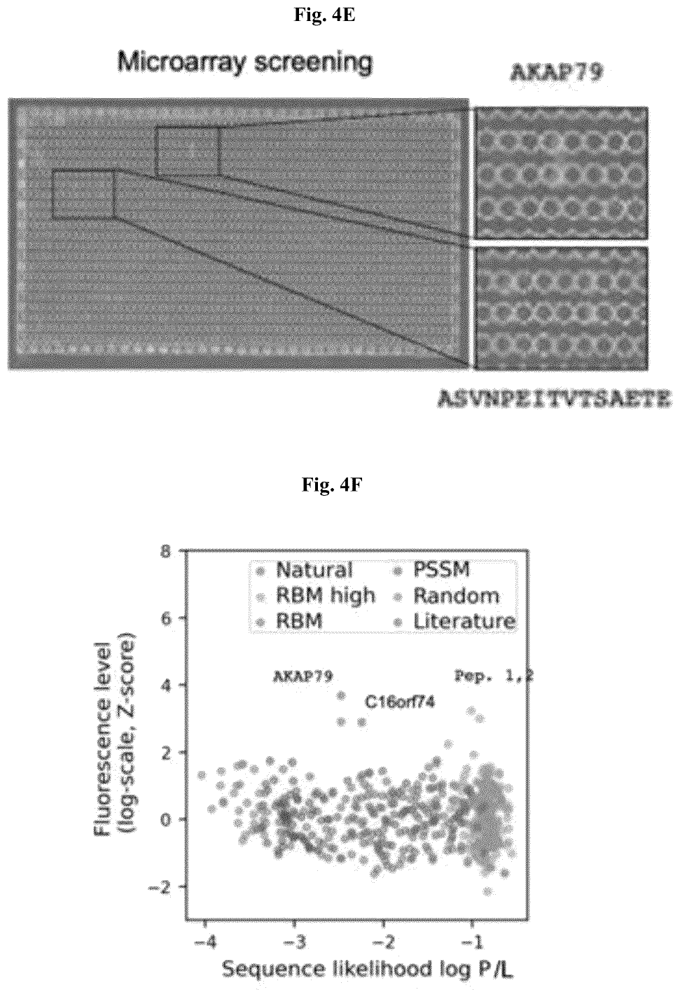

Next, at steps 210 and/or 212, screening is performed to identify the best candidates. The screening may include In-silico screening (Step 210) and/or in-vitro screening (Step 212). To this aim, at step 210, the binding strength (affinity) of the various candidate peptides to the target protein (Cn in this example) may be estimated in-silico by template-based docking followed by flexible backbone refinement using, for example, Modeller and PepCrawler (as shown, for example, in FIGS. 4A-F, detailed hereinbelow). In parallel, a medium-throughput qualitative binding assay may be performed at step 212, using suitable assays, such as, for example, using a PEPperPRINT peptide microarray to evaluate the direct binding of the target protein (i.e., Cn in this example), to selected peptides). The most promising peptides may then be selected at step 214, for further characterization.

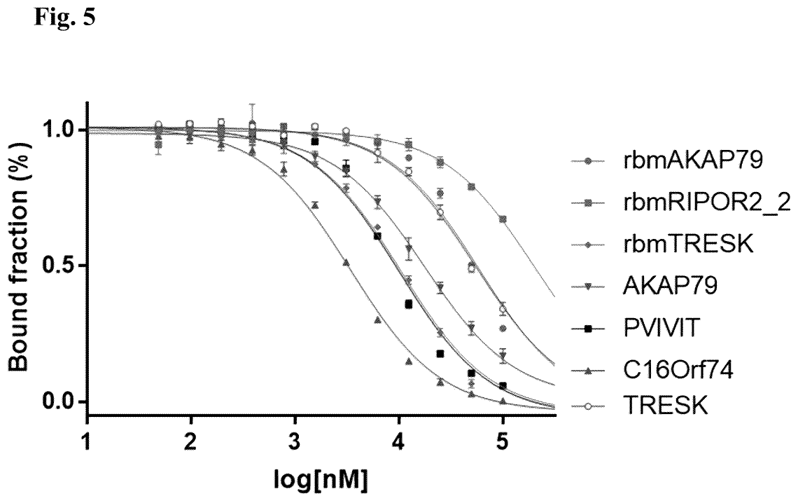

Next, at optional step 216, quantitative binding assay may be performed. To this aim, the ability of the designed peptides to compete with the binding of a control peptide (for example, PVIVIT peptide for Cn) may be experimentally quantified via suitable assays, such as, for example, Fluorescence Polarization (FP) assay (as shown, for example, in FIG. 5, detailed below herein).

According to some embodiments, at least some of the steps of the method are computerized.

According to some embodiments, for the sequence generative modeling, cRBMs may be trained on the multiple fragment alignment by Persistent Contrastive Divergence. In some embodiments, for the regularization, a sparse L₁² penalty may be used on the weights (of strength λ₁² ranging from 0.0 to 1.0) and a L₂ penalty may be used on the fields (of strength (log P_{θ}(S)). Training samples may be assigned a weight inversely proportional to the number of similar sequences in MSA with at most 1 similar amino acid. To calculate likelihood scores, the partition functions may be evaluated using the Annealed importance Sampling algorithm. To quantify the sparsity of the learnt sequence motifs, the fraction of non-zero weights may be estimated through participation ratios. For parameter selection, the MFA may be split into training and validation sets such that sequences from training and validation differed by at least three residues. This may be performed by hierarchical clustering. In some embodiments, after parameter selection, the best cRBM may be retrained over the full MFA.

According to some embodiments, the generative modeling may be an unsupervised learning modality. In some embodiments, it may include fitting a parametric probability distribution P_{θ}(S) over the sequence space by maximizing over the parameters θ the average likelihood (log P_{θ}(S) of observed sequences. Since P_{θ}(S) is normalized to unity

$${\sum }_{S} {P}_{Θ} ( S ) = 1 ,$$

this amounts to assigning large values of P_{θ}(S) for observed sequences and low elsewhere (This is exemplified in FIG. 3A). Thus, the learned P_{θ}(S) qualitatively reflects the evolutionary fitness function, which is also high for evolutionary selected sequences and low for unobserved sequences that were eliminated throughout evolution. Importantly, P_{θ}(S) must be “smooth” in sequence space, as the observed sequences only sparsely samples the set of all evolutionary fit sequences: unobserved but evolutionary fit sequences should also have high probability.

According to some embodiments, after training is completed, novel high-probability sequences distinct from the training data can be generated, and are potential target protein binders. The choice of functional form P_{θ}(S) determines the “smoothness” prior (i.e., the inductive bias) over the discrete sequence space. In some embodiments, the cRBM (as shown in FIG. 3B) may be used, formally defined as follows: Let S={s₁, . . . , sᵢ, . . . , s_{N}} be a protein sequence of length N, with sᵢ∈{A, C, D, . . . , Y, -} where - is the alignment gap symbol. Its probability P(S) writes:

$$P ( S ) = \frac{1}{Z} exp [ {\sum }_{i = 1}^{N} {g}_{i} ( {s}_{i} ) + {\sum }_{μ = 1}^{M} {Γ}_{μ} ( {\sum }_{i = 1}^{N} {ω}_{i μ} ( {s}_{i} ) ) ] \qquad ( 1 )$$

where Z is a normalizing factor (the partition function) such that Σ_{S} P(S)=1, gᵢ(n) are column-specific amino acid fields, W_{iμ} is a sparse weight matrix for projecting the sequence into a continuous, M-dimensional space (termed the hidden unit space) and the potentials Γ_{μ}(I) are trainable, strictly convex non-linearities (such as quadratic functions).

In some embodiments, informally, the fields quantify amino acid preferences at each column. High scores are assigned to sequences if their amino acids match the preferred ones at each location. Each weight vector w×μ informally represents a sequence motif consistently found in a subset of the data. The projection I_{μ}(S)=Σi w_{iμ}(sᵢ) quantifies the degree of matching between a given sequence and the motif, and the model allocates high probabilities to sequences that have either large positive or negative I_{μ}(S) via the quadratic-like non-linearity Γ_{μ}(I).

According to some embodiments, after training, novel sequences can be generated by combinatorial recomposition of positive and negative motif matches. In some embodiments, the cRBM was shown to be a powerful inductive bias for protein sequence modeling, as it generalizes over single-site and pairwise Potts models by incorporating sparse, high-order epistatic interaction terms and is easier to interpret than a pairwise model or deep generative models.

FIGS. 3A-H exemplify the generative modeling of PxIxIT binding motifs of Cn, as detailed below. FIG. 3A shows a schematic view of the generative approach: a “smooth” probability distribution over the whole sequence space is learnt from a limited number of samples. Unseen sequences with high probability are potential novel binders, whereas regions with low probability are likely non-functional proteins. FIG. 3B depicts the Restricted Boltzmann Machine (cRBM). FIGS. 3C and 3D provide the cRBM-predicted mutational landscapes for the NFATc2 and AKAP79 peptides. Red, white and blue entries correspond respectively to beneficial, neutral and deleterious mutations. FIG. 3E compares cRBM-predicted mutational landscapes with deep mutational scans of change in binding affinity measured by Nguyen et. al., (eLife. 2019 July; 8). Four DMS were performed taking as wild type the PVIVIT, PKIVIT, NFATc2 and AKAP79 peptides. FIGS. 3F, 3F, and 3H show selected examples of sequence motifs learnt by the cRBM, as detailed below. Shown are the sequences (FIG. 3F), their activity distribution (FIG. 3G) and top-activating sequences (FIG. 3H). In the example shown in FIG. 3H, Motif 1 is gene-specific, whereas motifs 2 and 3 are shared by multiple genes. Mutational landscapes may be similarly predicted for the designed and experimentally characterized peptides, and the beneficial and neutral mutations may be used to define the consensus sequences. The presented sequence motifs show what is learned by the model, but they can differ from the defined consensus.

According to some embodiments, the sequence model a priori may treat all natural sequences equally. However, their binding affinities span almost may span several orders of magnitude (for example, 0.5-250 μM). Thus, to further refine the list of candidate peptides, the docking energy score may be estimated (where a lower score is better) using, for example, crystal structures of target protein bound to a known motif binding peptide and an ad-hoc template-based molecular docking followed by a flexible-backbone refinement pipeline based on Modeller (52) and/or PepCrawler may be applied. According to some embodiments, to rationalize the docking energy score from the peptide sequence, an additive single-site model may be fitted to the docking results by sparse linear regression.

According to some embodiments, to evaluate the ability of docking energy to discriminate between natural binders, approximate docking scores may be predicted for all natural fragments using, for example, a single-site model and a per-substrate average may be computed.

According to some embodiments, the docking score may efficiently complement the evolutionary score by differentiating between natural genes with variable activation levels.

According to some embodiments, for the construction of multiple fragment alignment of natural binders, various initial seed alignments of interacting orthologs may be first constructed. Orthologs may be collected from various sources, such as, for example, the Homologene database, a BLAST search over a database (such as, UniProt, UniClust30 database, etc.). The seed sequences may be aligned using suitable tools, such as, MAFFT, KMAD, and the like. Non interacting homologs may be filtered out. For example, if the interaction between protein 1 and 2 is conserved in species A and B, then their sequences should have diverged at a similar rate from one another Sim(P₁^{A}, P₁^{B})∝Sum(P₂^{A}, P₂^{B}). Conversely, deviations from this pattern indicate possible gene duplication events that do not necessarily preserve functional interaction, such that duplicates may be removed, and a single copy of target protein subunit may be identified for each species. Next, for each interacting substrate protein (SP), its presumed interacting fragment may be extracted by taking all amino acids (including insertions) at designated location of the Short Linear Motif (SLIM) for the seed sequences. The fragments may be pooled together, and realigned. At this stage, fragments that clearly deviated from the main distribution (abnormally long, no visible SLIM), may be removed. To this aim, a Restricted Boltzmann Machines may be trained, and the likelihood log P may be computed for each sequence. In some embodiments, to determine a cut-off, the sequences may be grouped, and a corresponding sequence profile may be computed for each group. Sequences with Z-normalized likelihood score below a designated threshold (e.g., those that do not feature the expected SLIM motif, nor any significant sequence conservation) may be discarded. After filtering, realignment and retraining may be performed.

According to some embodiments, the method for identification of peptides binding target protein-protein interaction surface may utilize the following inputs:
1. A target protein
2. A list of binding fragments extracted from known protein binders of the target. For example, previously elucidated in previous studies via high-throughput search (e.g., yeast display).
3. One or more experimental/model structure of the target binding fragment complex, for one of the above fragments

According to some embodiments, the method for identification of peptides binding target protein-protein interaction surface may utilize the output of: a list of candidate binding peptides, with predicted evolutionary likelihood and binding scores.

According to some embodiments, the method may include one or more of the steps of: Constitution of a set of natural binding fragments; Training, validation of a sequence generative model and generation of artificial sequences; Scoring of designed peptides by template-based flexible docking; and Designed peptide selection.

According to some embodiments, in order to enhance the outcome of disclosed methods one or more of the following characteristics may be applicable:
i) Training the SGM may require a sufficiently diverse set of sequences. Thus, the protocol is in particularly applicable if the interaction is highly conserved throughout evolution, and/or if multiple natural binders have been characterized;
ii) Pairing of interacting orthologs may be challenging and accordingly, there is no guarantee that all sequences in the multiple fragment alignment will indeed bind the target;
iii) An experimental structure or reliable model should be available for at least one binder in order to perform template-based docking and scoring;

According to some embodiments, there is provided a system for the design of peptide inhibitors of a target PPI, the system includes a processing circuitry; and a memory, the memory containing instructions that, when executed by the processing circuitry, configure the system to execute the method as disclosed herein.

According to some embodiments, there is provided a non-transitory computer readable medium having stored thereon instructions for causing a processing circuitry to execute the method for the design of peptide inhibitors of a target PPI, as disclosed herein.

Unless defined otherwise, all technical and scientific terms used herein have the same meaning as commonly understood by one of ordinary skill in the art to which the invention pertains.

The term “gene”, as used herein, may refer to the actual genomic gene (DNA sequence), but may also be used to refer to the protein encoded by the gene (amino acid sequence), according to context.

The term “a” and “an” refers to one or to more than one (i.e., to at least one) of the grammatical object of the article. By way of example, “an element” means one element or more than one element.

The term “about” when referring to a measurable value such as an amount, a ratio, and the like, is meant to encompass variations of ±10% of the indicated value, as such variations are also suitable to perform the disclosed invention. Any numerical values appearing in the application are intended to be construed as if preceded by “about”, unless indicated otherwise.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The present invention may be a system, a method, and/or a computer program product. The computer program product may include a computer readable storage medium (or media) having computer readable program instructions thereon for causing a processor to carry out aspects of the present invention.

A computer program (also referred to as a program, software, software application, script or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A computer program can be stored in a portion of a file that holds other programs or data, in a single file dedicated to the program in question, or in multiple coordinated files (for example, files that store one or more modules, sub programs or portions of code). A computer program can be deployed to be executed on one computer or on multiple computers that are located at one site or distributed across multiple sites and interconnected by a communication network.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, for example, JavaScript, Smalltalk, C, C++, TypeScript, Python and R.

The computer readable program instructions may execute entirely on the user's computer, partly on the user's computer, as a stand-alone software package, partly on the user's computer and partly on a remote computer or entirely on the remote computer or server (such as, a cloud based). In the latter scenario, the remote computer (or cloud) may be connected to the user's computer through any type of network, including a local area network (LAN) or a wide area network (WAN), or the connection may be made to an external computer (for example, through the Internet using an Internet Service Provider) including wired or wireless connection (such as, for example, Wi-Fi, BT, mobile, and the like). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention. Moreover, a computer can be embedded in another device, for example, a mobile phone, a tablet, a personal digital assistant (PDA, or a portable storage device (for example, a USB flash drive). Devices suitable for storing computer program instructions and data include all forms of non-volatile memory, media and memory devices, including semiconductor memory devices, for example, EPROM, EEPROM, random access memories (RAMs), including SRAM, DRAM, embedded DRAM (eDRAM) and Hybrid Memory Cube (HMC), and flash memory devices; magnetic discs, for example, internal hard discs or removable discs; magneto optical discs; read-only memories (ROMs), including CD-ROM and DVD-ROM discs; solid state drives (SSDs); and cloud-based storage. The processor and the memory can be supplemented by, or incorporated in, special purpose logic circuitry.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The processes and logic flows described herein may be performed in whole or in part in a cloud computing environment. For example, some or all of a given disclosed process may be executed by a secure cloud-based system comprised of co-located and/or geographically distributed server systems. The term “cloud computing” is generally used to describe a computing model which enables on-demand access to a shared pool of computing resources, such as computer networks, servers, software applications, and services, and which allows for rapid provisioning and release of resources with minimal management effort or service provider interaction.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments.

While certain embodiments of the invention have been illustrated and described, it will be clear that the invention is not limited to the embodiments described herein. Numerous modifications, changes, variations, substitutions, and equivalents will be apparent to those skilled in the art without departing from the spirit and scope of the present invention as described by the claims, which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Materials and Methods

Construction of multiple fragment alignment of natural binders. The following abbreviations are used: Catalytic subunit of CaN: CaNA, Regulatory subunit of CaN: CaNB, substrate protein: SP. Short Linear Motif: SLIM.

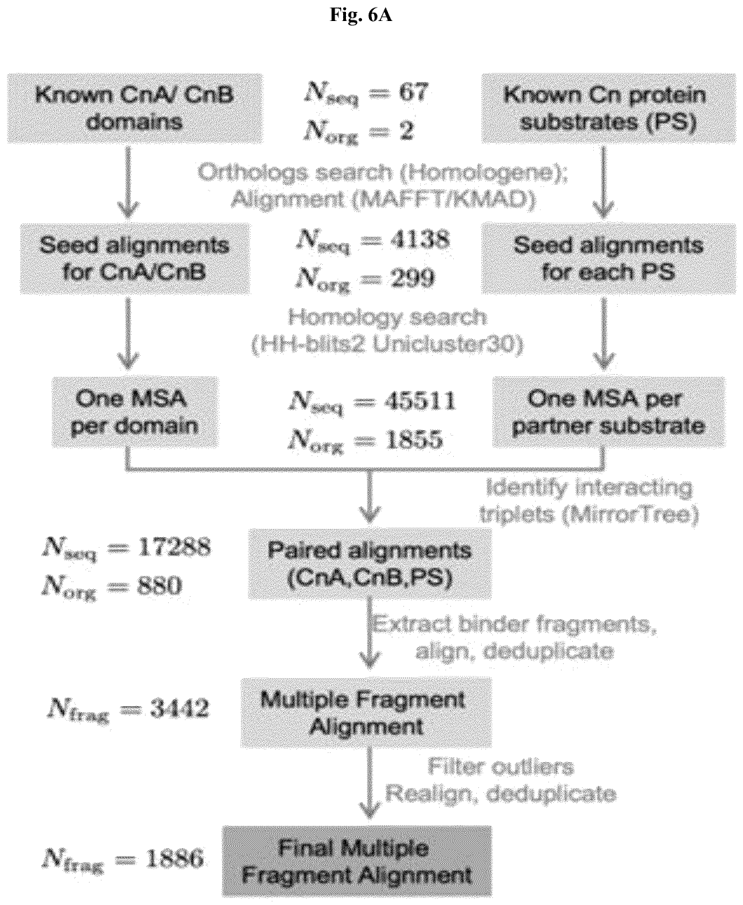

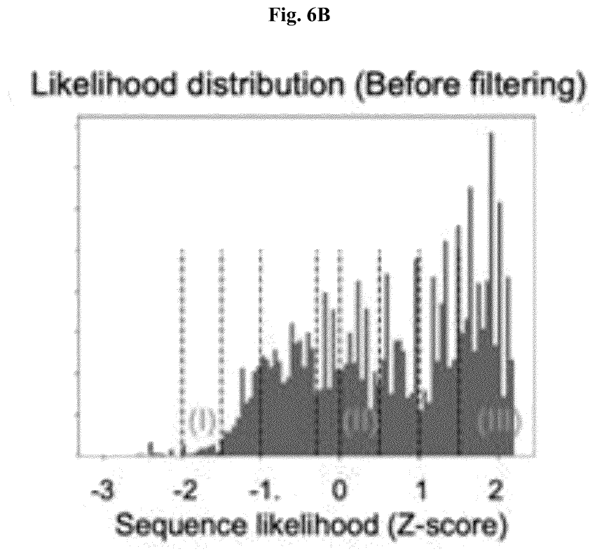

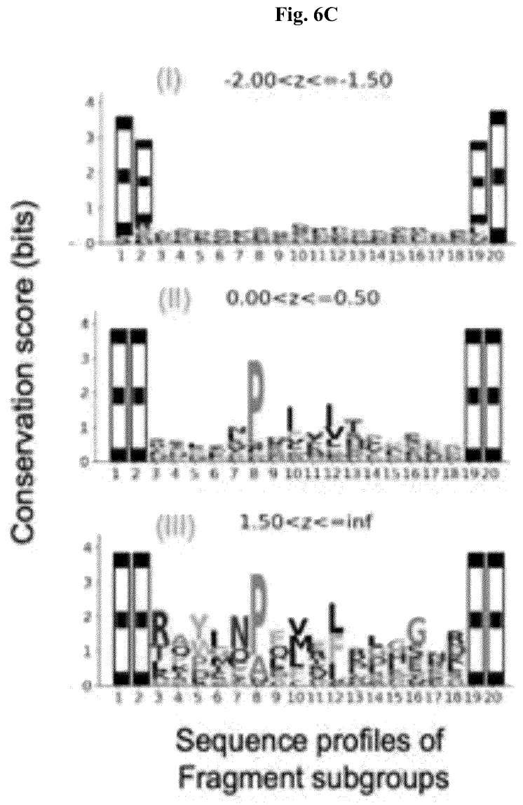

A flow chart summarizing the alignment protocol is shown in FIG. 6A. The protocol initiates from a list of 67 (38 from yeast, 29 from human) experimentally validated substrate proteins of the two subunits CaN enzyme (UniProt accession numbers CaNA—Q08209, CaNB—P63098). For each SP, the location of the fragment binding the CaN PxIxIT binding site was identified by phage display screening and/or SLIM matching. Since generative modeling generally benefits from increased sequence diversity, this initial set was augmented by homology search. However, naive homology search was not suitable because CaN-SP interactions are not systematically conserved throughout evolution; therefore, the protocol instead proceeded as follows.

For CaNA, CaNB and for each SP, initial seed alignments of interacting orthologs were first constructed. Orthologs were collected from the Homologene database if available, or via a BLAST search over UniProt (default parameters, top-100 hits, keeping only sequences with identical or synonymous gene name). For ordered proteins, the seed sequences were aligned using MAFFT (default parameters). For disordered proteins (identified as such by IUPred2), visual inspection of MAFFT outputs revealed unsatisfactory alignments that did not consistently align the binding SLIMs. Instead, KMAD, a multiple alignment software tailored for disordered proteins was used (parameters: custom pattern “P.I.I.”, add score=100, subtract score=5, default otherwise).

Next, for each CaN subunit and SP, additional homologs were searched over the UniClust30 database (time stamp: 2018/06) using HHblits 3 (4 iterations, default values of other parameters).

Non-interacting homologs were next filtered out using a variant of the MirrorTree approach. The intuition behind MirrorTree is that when two protein families interact, their respective phylogenetic trees tend to be similar. More specifically, if the interaction between protein 1 and 2 is conserved in species A and B, then their sequences should have diverged at a similar rate from one another Sim(P₁^{A}, P₁^{B})∝Sum(P₂^{A}, P₂^{B}). Conversely, deviations from this pattern indicate possible gene duplication events that do not necessarily preserve functional interaction. Formally, there is for each SP a set of seed triplets [(CaNA^{SeedOrg1}, CaNB^{SeedOrg1}, SP^{SeedOrg1}), (CaNA^{SeedOrg2}, CaNB^{SeedOrg2}, SP^{SeedOrg2}), . . . ] and a set of candidate triplets [(CaNA^{Org1}, CaNB^{Org1}, SP^{Org1}), (CaNA^{Org2}, CaNB^{Org2}, SP^{Org2}), . . . ] to be filtered out—in general there are multiple candidate triplets per organism. For each triplet [symbol] and seed triplet l′ the sequence identity of each partner is computed:

$${S}_{l , {l}^{′} , k} = Sim [ {P}_{k}^{l} , {P}_{k}^{{l}^{′}} ]$$

where k∈(CnA, CnB, SP) is the complex component index.

Next, the Pearson correlation matrix was determined as,

R_{I,k,k′}=PearsonR_{P}(S_{l,l′,k}) followed by its off-diagonal average: Rₗ−⅓(R_{l,CnA,CnB}+R_{l,CnA,SP}+R_{l,CnB,SP}). R quantifies the overall consistency of the evolutionary divergence of the proposed triplet with respect to the seed triplets.

Next, a single copy of CaNA and CaNB was identified for each species by maximizing the average Rₗ. Triplets involving another CaNA/CaNB copy were discarded, and among the remaining ones all triplets with Rₗ above some threshold were kept. The threshold was determined individually for each SP, as the minimum over seed triplets of Rₗ (i.e., such that all seed triplets were kept).

Next, for each interacting SP, its presumed interacting fragment was extracted by taking all amino acids (including insertions) between columns i*−10 and I*+16, where i* is the starting location of the SLIMS for the seed sequences. The fragments were pooled together, and realigned with MAFFT (gap opening penalty: 6, gap extension penalty: 2). At this stage, visual inspection revealed fragments that clearly deviated from the main distribution (abnormally long, no visible SLIM), presumably because of sequence alignment errors or not being properly filtered out.

To remove these sequences, a Restricted Boltzmann Machine (RBM) with 10 dReLU hidden units and sparse regularization penalty

$${λ}_{1}^{2} = {10}^{- 2}$$

(as detailed below) was trained, and the likelihood log P was computed for each sequence. The distribution, shown in FIG. 6B featured a heavy left tail, meaning that many sequences were outliers, belonging to sparsely populated regions of the sequence space.

To determine a cut-off, the sequences were grouped by likelihood interval (dotted lines), and a sequence profile was computed for each group (FIG. 6C). Sequences with Z-normalized likelihood score below −0.3 did not feature the expected SLIM motif, nor any significant sequence conservation, and were therefore discarded. After filtering, realignment and retraining, the new likelihood distribution (not shown) featured a unimodal shape consistent with previously studied models of protein families, and the sequence profile featured the expected conservation patterns. Finally, only 5 flanking residues were retained on each side to facilitate comparison with previous works. Note that we did not use the CaNA/CaNB alignments, as the binding site to PxIxIT was highly conserved and there was no coevolution between CaN and its substrates.

Sequence generative modeling. cRBMs were trained on the multiple fragment alignment by Persistent Contrastive Divergence following Tubiana J, et. al., (eLife. 2019 Mar. 12; 8:039397). The algorithm was implemented in Python3.8, using mainly numpy and numba (source code available from https://github.com/jertubiana/PGM), and the following parameters were used: number of hidden units: from 5 to 30; hidden unit potential: double Rectified Linear Units (dReLU); batch size: 100; MCMC sampler: alternate Gibbs; number of Markov chains: 100; number of Monte Carlo steps between each gradient evaluation: 20; number of gradient updates: 20000; optimizer: ADAM with initial learning rate: 10⁻³, exponentially decaying after 50% of the training to 10⁻⁵, β₁=0, β₂=0.99, ϵ=10⁻³.

For the regularization, a sparse penalty was used on the weights (of strength λ₁² ranging from 0.0 to 1.0) and a L₂ penalty was used on the fields (of strength (log P_{θ}(S)). Training samples were assigned a weight inversely proportional to the number of similar sequences in MSA with at most 1 similar amino acid.

To calculate likelihood scores, the partition functions were evaluated using the Annealed importance Sampling algorithm, using 10⁴ intermediate temperatures and 10 repeats. To quantify the sparsity of the learnt sequence motifs, the fraction of non-zero weights was estimated through participation ratios as described in Eqn. 20,21 of Tubiana J, et. al. For parameter selection, the MFA was split into training and validation sets such that sequences from training and validation differed by at least three residues. This was done by performing hierarchical clustering with single linkage merging criterion (scipy.cluster.hierarchy.single command), cutting the tree at 2 and assigning 80% of the clusters to train and 20% to validation.

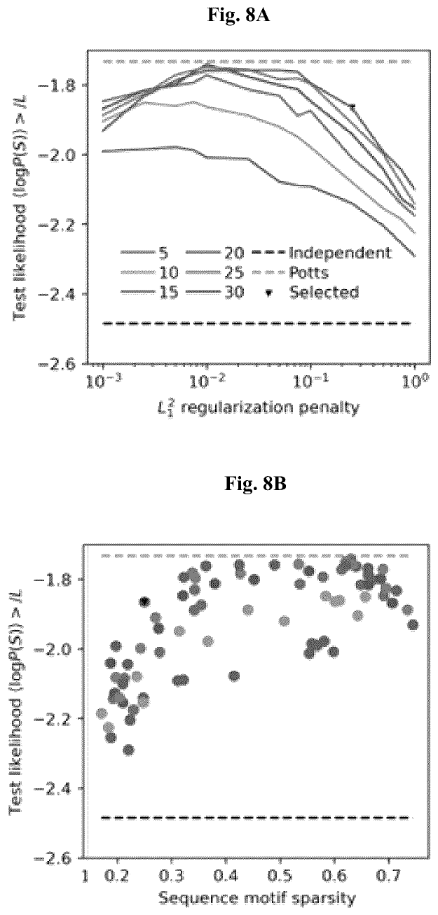

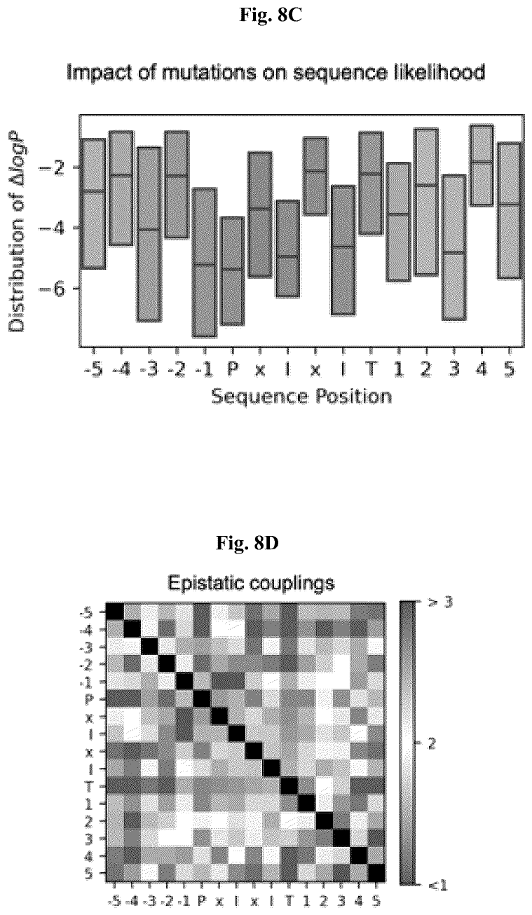

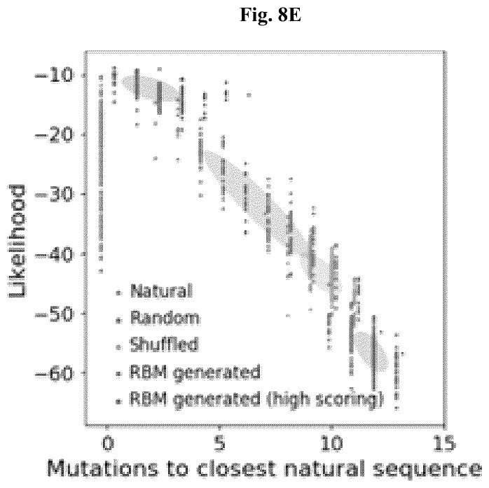

A grid search was performed over the number of hidden units and the regularization strength, and the model sparsity and held-out average log-likelihood were monitored (FIG. 8A, FIG. 8B); the model that best compromise between good sparsity and likelihood was selected, corresponding to 30 hidden units and 0.25λ₁² regularization strength. Its per-site likelihood was substantially better than the best independent/position-specific scoring matrix (PSSM) model (optimized over pseudo-count value) and slightly lower than the best Potts model trained using the same algorithm (optimized over regularization strength).

After parameter selection, the best cRBM was retrained over the full MFA. After training, mutational landscapes shown in FIGS. 3C and 3D were computed by repeated application of the equation (1) for wild-type and single-point variants. The effective epistasis matrix of FIG. 3D was computed via Eqn 15,16 of Tubiana J, et. al. Artificial sequences were synthesized by MCMC sampling with an alternate Gibbs sampler, 1000 burn-in MC steps and 100 MC steps between each sample. Both regular sampling and low-temperature sampling (i.e. sampling from ΔΔG were used, so as to generate sequences with high likelihood. Low-temperature sampling was implemented via the duplicate RBM trick (described in Eqn. 14 of Tubiana J, et. al.). Roughly 10 000 peptides were sampled for regular and low-temperature sampling, and hierarchical clustering was subsequently performed to reduce it to 361 and 180 peptides (one representative that maximizes the likelihood per cluster).

Based on entropy calculations of the cRBM distribution and low-temperature cRBM distribution, the size of the set of CaN-binding peptides was estimated to be 10^{13.7} and 10^{2.8} respectively—a tiny fraction of the 10^{20.8} possible peptides of length 16.

Template-based docking and binding scoring. A physical binding score was determined for each of the 768 candidate peptides by template-based docking as follows. Five structures of CaN catalytic subunit in complex with various PxIxIT-containing peptides were collected from the pdb: 2p6b (PVIVIT), 3118 (AKAP79), 6uuq (RCAN1), 6nuf (NHE1) and 2jog (PVIVIT, NMR). Given a peptide sequence and template complex, the candidate and template peptide sequences were aligned (by motif matching), then 100 homology structural models for the peptide were built using Modeller. The candidate peptide was superimposed onto the template peptide and translated away from CaN if steric clashes occurred (<2A center-center distance between any pair of atoms).

Then, a model in extended conformation and forming many contacts was selected by maximizing the cost function Coverage+0.05*NumAtomContacts+0.05*Extension where NumAtomContacts is the number of atomic contacts (heavy atoms only, 4A distance cutoff between atom centers), Coverage is the number of peptide residues forming at least one atomic contact and Extension is the euclidean distance between the C-terminal and N-terminal C-alpha atoms.

Next, the initial conformation was refined using PepCrawler conformational sampling algorithm based on rapidly-exploring random trees and an all-atom energy function. The above homology modeling+refinement protocol was repeated 10 times for each structure and the configuration with minimum energy was retained. In total, each peptide was docked 50 times, corresponding to ˜1-2 days of computation on a single CPU core of an Intel Xeon Phi processor. The funnel score—a measure of the steepness of the energy landscape around the minimal energy configuration—was also computed to characterize good peptide inhibitors The docking energies correlated well (r˜0.5 for all pairs) but not the funnel scores, allegedly due to the homology modeling step or to the relatively long peptide length. Thus, only the binder energy score was retained.

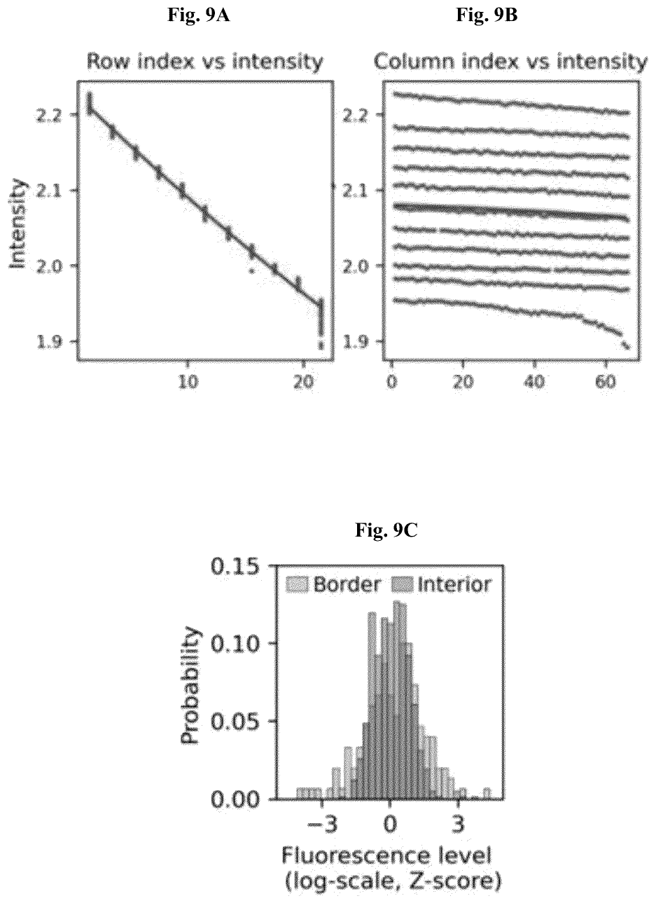

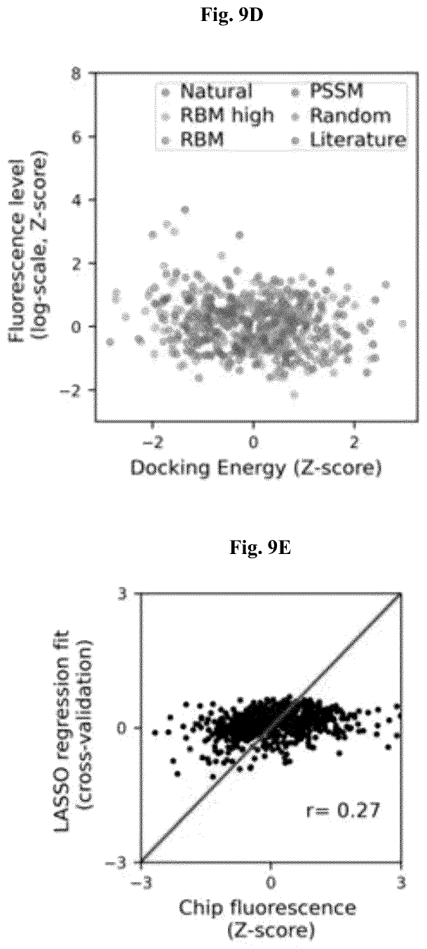

Peptide array readout and analysis. The peptide array was prepared as a custom array pepper chip (PEPperPRINT®). For binding detection, 1 mg/ml GST-tagged CaN was incubated overnight on the microarray at 4° C. Following extensive washing, fluorescently labeled (Alexa-Fluor 647) GST-antibody. After additional washing and drying according to suggested manufacturer protocol, the microarray slide was scanned with an InnoScan 1100 scanner (Innopsys). The experiment was repeated five times. Each scan was analyzed as follows: a grid was overlaid using the border HA markers to determine regions of interest (ROI) for each peptide (two ROIs per peptide) and the logarithm of the average fluorescence intensity was computed for each ROI. The baseline fluorescence level was not uniform throughout the array as evidenced from scatter plots of log-fluorescence intensity against row and column index (FIGS. 9A and 9B).

To remove spatial artifacts, a position-dependent baseline fluorescence was fitted using a second-order polynomial, and subtracted to the fluorescence. Fluorescence levels were next averaged over the two ROI for each peptide and Z-normalized. The peptides lying along the border were found to have significantly higher fluorescence level due to oversplash from the border HA markers (c) and were not further analyzed. It was determined that there was no significant oversplash within the interior of the chip by monitoring the spatial autocorrelation function of fluorescence scores. The Z-scores were averaged over the five repetitions to yield one fluorescence score per peptide.

CaN expression and purification. The catalytic subunit of CaN was expressed as described in Gal M, et. al., (Structure. 2014 Jul. 8; 22 (7): 1016-27). Briefly, the gene encoding residues 2-347 of CaNA (UniProt accession Q08209) with substitutions Y341S, L343A, and M347D was expressed as a cleavable GST fusion protein in E. coli BL21 cells. Growing in LB, after reaching to OD (600 nm)=0.8, protein expression was induced by the addition of 1 mM IPTG at 25° C. The cells were harvested after 16 h and resuspended in PBS-based lysis buffer suitable for downstream purification onto GST column (Glutathione Sepharose 4 Fast Flow) of the soluble fraction after disrupted by sonication and remove of all non-soluble debris by centrifuge. Elution from the GST column was further purified by size exclusion chromatography with the superdex 75 column.

Peptide synthesis. Peptides were synthesized with N-ter acetylation and C-ter amidation by peptide2 Inc (USA).

Fluorescence Polarization (FP) competition assay. Fluorescence measurements were performed on samples arrayed in a 96-well plate using Biotek HybridHI reader equipped with a polarized optic system. All measurements were done in triplicate. Competition was evaluated by adding variable concentrations of each non-labeled tested peptide to wells containing 100 nM of FITC-labeled PVIVIT peptide and 4 uM of CaNA. Experimental polarization data from simple and competitive binding experiments were fitted using GraphPad Prism7, with error bars representing standard deviation.

Post-hoc analysis. All statistical analysis, including statistical tests, LASSO regression and T-SNE dimensionality reduction were performed using the numpy, scipy and scikit-learn Python packages.

### Example 1: The CaN Signaling Network Relies on the PxIxIT and LxVP SLIMs

Calcineurin (CaN) is a heterodimeric calcium-dependent phosphatase conserved in metazoans, constituted by a catalytic (˜510 amino acids) and a regulatory subunit (˜170 amino acids), see structure is shown in FIG. 1A. Upon calcium chelation and interaction with calmodulin (both mediated by the regulatory subunit), CaN adopts its active conformation in which its catalytic site and binding regions are exposed. In turn, CaN substrates—most of which are intrinsically disordered—bind it, enabling dephosphorylation of serine and threonine residues by CaN. The NFAT family—a set of five transcription factors conserved in vertebrates—are known examples of substrate of CaN. Upon dephosphorylation by CaN, they undergo conformational changes that expose nucleus localization motifs, allowing translocation to the nucleus, and in turn, binding to DNA. More generally, the CaN signaling network was systematically investigated in mammals and yeast using combinations of in-vivo, in-vitro and in-silico methods, and at least 29 and 38 protein substrates were identified with high confidence respectively for human and yeast.

To determine the regions tethering the substrates, the ScanNet web server was used to predict binding sites of intrinsically disordered proteins. In addition to the catalytic site, two substrate binding sites are found. Two SLIMs were identified in previous studies: PxIxIT and LxVP, where uppercase letters stand for conserved residues and x represents alternate amino acids. Both motifs: i) bind CaN in isolation (crystal structures of representative CaN-bound PxIxIT and Lx VP motifs are depicted in respectively magenta and yellow of FIG. 1A); and ii) are conserved across a wide range of substrates as illustrated for the NFAT isoforms in FIG. 1B.

Substrate-derived, PxIxIT-containing fragments bind relatively weakly to CaN, with dissociation constants k_{d}˜0.5-250 uM. Indeed, higher affinity interactions may be deleterious in vivo. For example, the CaN-NFAT interaction is evolutionarily tuned to occur only at high calcium concentrations. This pushed the design of PxIxIT peptide variants with higher affinity-such as the PVIVIT peptide (k_{d}˜0.5-2.0 uM) and its peptidomimetics derivatives (up to k_{d}˜2.5 nM) —that can successfully outcompete CaN-substrate binding in the cell and hence dephosphorylation. However, these peptides were mostly discovered experimentally based on the limited sequential space of NFAT-derived peptides, without exploring the vast range of additional substrates motifs.

The present strategy pursued according to the disclosure aimed to design peptides capable of competing with the known PVIVIT peptide. New peptide sequences pave the way for further design of variants with higher affinity, specificity, and/or solubility than previous sequences.

FIGS. 1A-B summarize the Calcineurin-NFAT complex. FIG. 1A illustrates the structure of Calcineurin bound to representative SLIM-containing peptides (pdb codes: 5sve, 2p6b). Calcineurin is shown in molecular surface representation, and is colored by propensity to bind disordered regions (from white=low to dark red=high), based on ScanNet. The catalytic site is colored in blue. Both the catalytic and the regulatory (circled in green) subunits are shown. PxIxIT and LxVP-containing peptides are shown in stick representation (resp. in magenta and yellow). FIG. 1B provides the sequence alignment of the PxIxIT short linear motifs that bind calcineurin.

### Example 2: Cn Binding Peptide Design Protocol

Curation of known CaN-binding fragments from literature survey. The protocol started with a list of 67 protein substrates of CaN from human and yeast that have been previously characterized, together with their corresponding PxIxIT-containing fragment(s).
Data augmentation by homology search. Since this number is too limited for meaningful SGM, the set was first enriched by performing a homology search across sequence databases to identify additional PxIxIT-like fragments in homologous sequences for each of the listed substrates. Importantly, the PPI is not guaranteed to be conserved across all orthologs/paralogs, especially in cases like the CaN signaling networks, which undergo rapid rewiring throughout evolution. Therefore, a two-stage sequence-based statistical filtering protocol is applied to eliminate presumed non-interacting homologs. After realignment and deduplication, a multiple sequence alignment of natural, putatively CaN-binding fragments was obtained.

Peptide library design using a SGM-Next, a compositional Restricted Boltzmann Machine (cRBM) was trained. A cRBM is an interpretable SGM inspired by statistical mechanics and previously shown to be suitable for protein sequence modeling. After quantitatively and qualitatively validating the model learned (FIGS. 3A-H and FIGS. 8A-E), it was used to generate a diverse library of 10²⁻³ candidate peptide sequences. Negative and positive controls were also constructed for subsequent quality assessment.
In-silico and in-vitro screening. Then the binding strength of the various peptides to CaN was estimated in-silico by template-based docking followed by flexible backbone refinement using Modeller and PepCrawler (FIGS. 4A-F). In parallel, a medium-throughput qualitative binding assay was performed using a PEPperPRINT peptide microarray to evaluate the direct binding of CaN to selected peptides (FIGS. 9A-E). The most promising peptides were selected for further characterization.
Quantitative binding assay. Finally, the ability of the designed peptides to compete with the binding of PVIVIT peptide for CaN were experimentally quantified via Fluorescence Polarization (FP) assay (FIG. 5).

### Example 3: Natural CaN-Binding Peptides are Highly Diverse

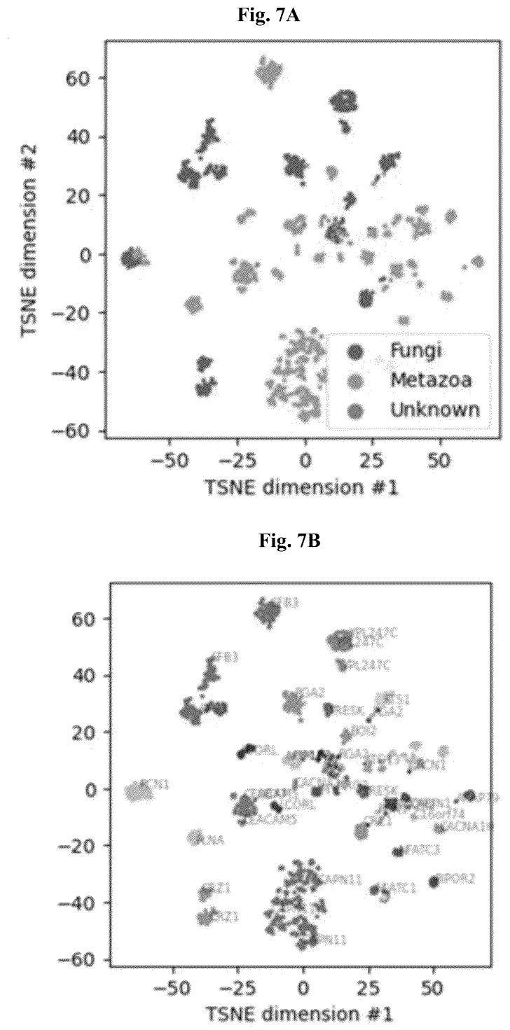

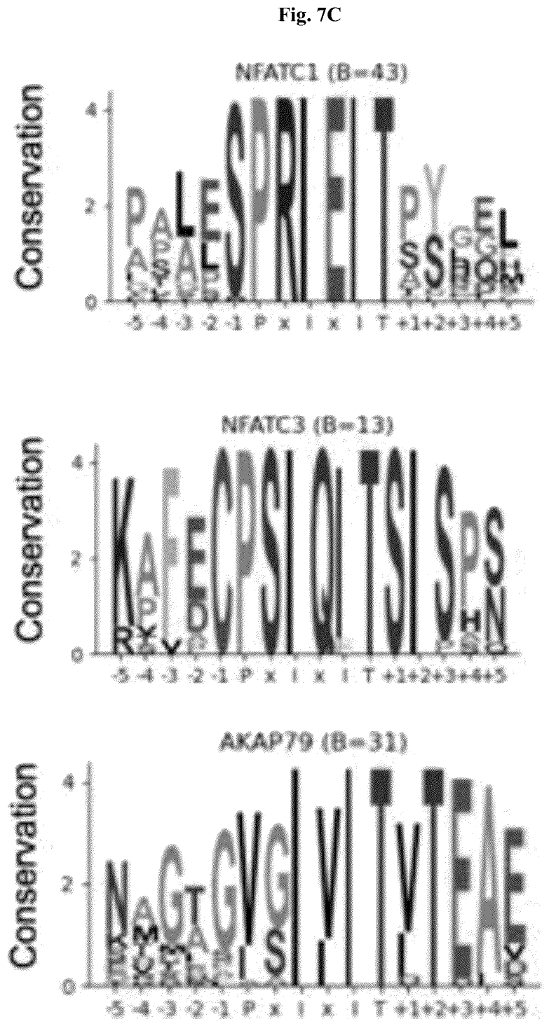

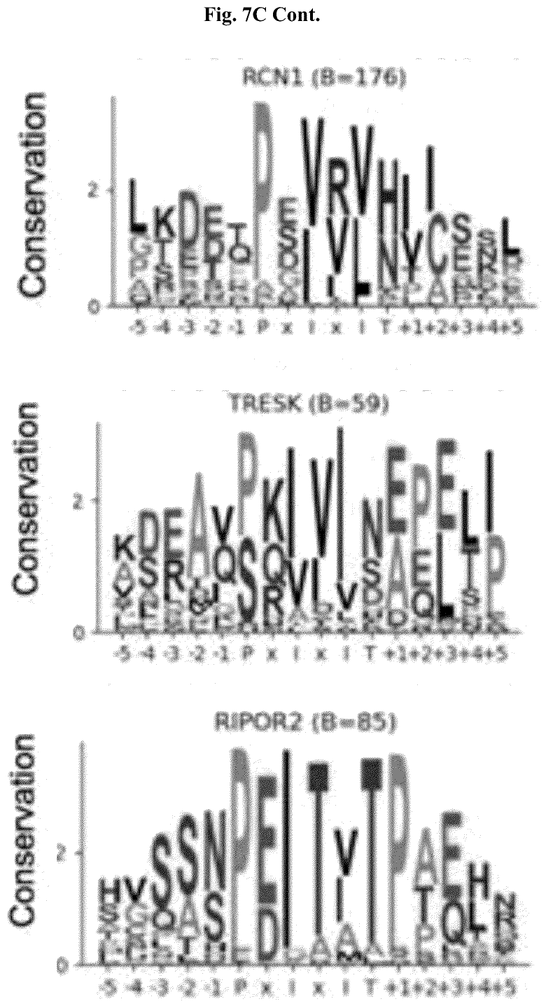

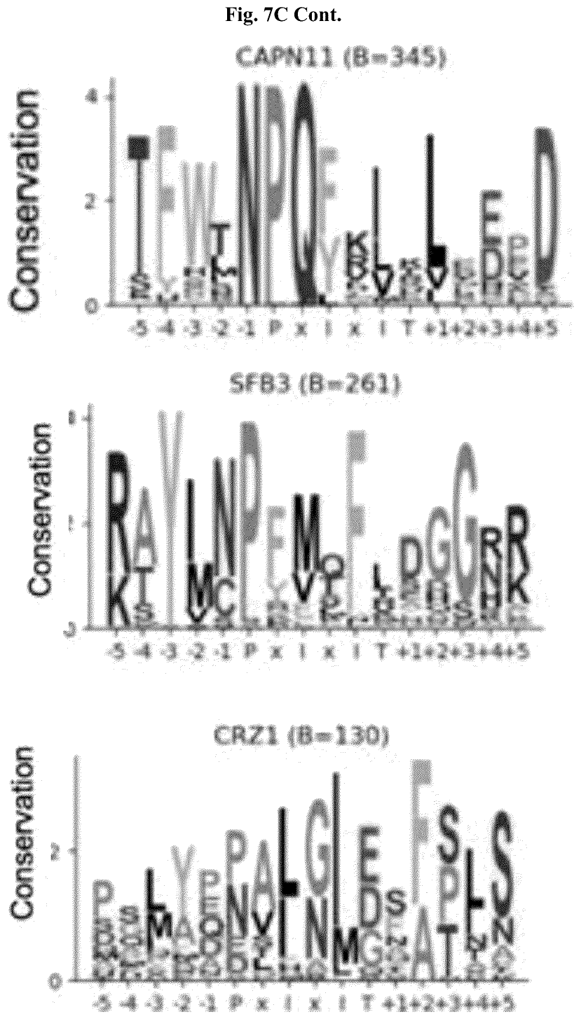

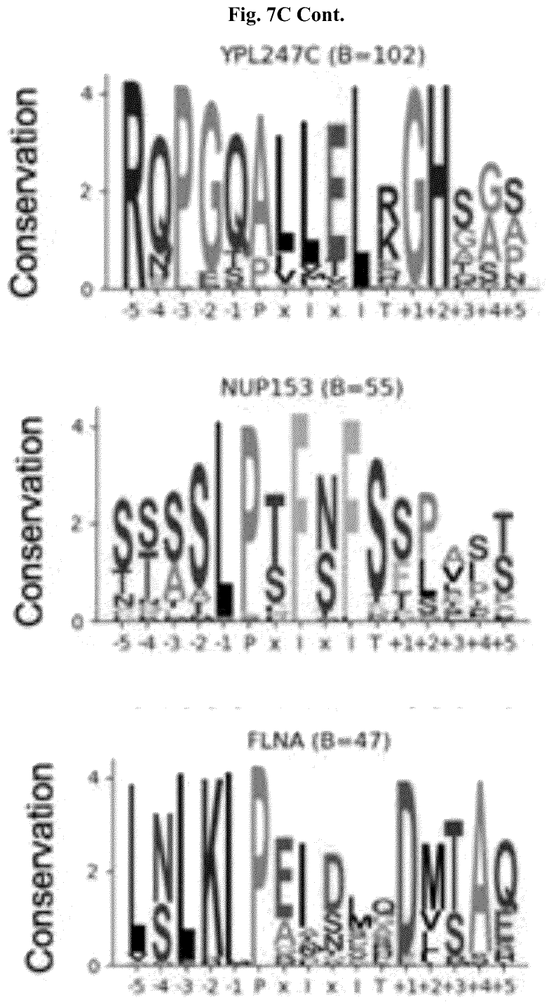

After the homology search, a multiple alignment of 1886 fragments and 16 columns were obtained, corresponding to the six motif positions and five flanking residues on each side. Sequence logo visualization (FIGS. 7A-C) revealed high sequence diversity: strong amino acid preferences were only observed at positions P, I₁ and I₂, respectively for proline and hydrophobic residues. T-SNE projections showed that fragments mostly cluster by source gene and taxon (FIG. 7B, FIG. 7C). Sequence logo visualization of individual gene sub-alignments revealed stronger conservation patterns). The central SLIMs are more diverse than PxIxIT, e.g. gene-specific SLIMs P[T/S]F[N/S]FS, P[E/D]IT[V/I]T, PQ[F/Y]x[I/L/V]x, P[F/Y][M/V]xFx are found. Conservation patterns are also found outside of the six canonical SLIM positions: e.g. for the CAPN11 gene, positions −5, −4, −1, +1, +5 are conserved, suggesting that they are also important for binding.

In summary, natural CaN binders have diverse sequences that are not well recapitulated by a single SLIM or PSSM model. Such a combination of local conservation and global diversity may have arisen from multiple binding conformations and/or distinct spatial repartition of the binding energy. Recombining these motifs may yield synthetic sequences with similar or improved binding compared to their natural counterparts. Moreover, it may enable specific competition with a defined substrate, while maintaining binding for others.

### Example 4: Reverse Engineering of Binding Fragments by SGMs (Step 2)

Generative modeling is an unsupervised learning modality. It consists of fitting a parametric probability distribution P_{θ}(S) over the sequence space by maximizing over the parameters (θ) the average likelihood [symbol] log P_{θ}(S) [symbol] of observed sequences. Since P_{θ}(S) is normalized to unity

$${\sum }_{S} {P}_{Θ} ( S ) = 1 ,$$

this amounts to assigning large values of P_{θ}(S) for observed sequences and low elsewhere (FIG. 3A). Thus, the learned P_{θ}(S) qualitatively reflects the evolutionary fitness function, which is also high for evolutionary selected sequences and low for unobserved sequences that were eliminated throughout evolution. Importantly, P_{θ}(S) must be “smooth” in sequence space, as the observed sequences only sparsely samples the set of all evolutionary fit sequences: unobserved but evolutionary fit sequences should also have high probability.

After training is completed, novel high-probability sequences distinct from the training data can be generated, and are potential CaN binders. The choice of functional form P_{θ}(S) determines the “smoothness” prior (i.e., the inductive bias) over the discrete sequence space. Here, the cRBM (FIG. 3B) were used, formally defined as follows. Let S={s₁, . . . , sᵢ, . . . , s_{N}} be a protein sequence of length N, with sᵢ∈{A, C, D, . . . , Y, -} where - is the alignment gap symbol. Its probability P(S) writes:

$$P ( S ) = \frac{1}{Z} exp [ {\sum }_{i = 1}^{N} {g}_{i} ( {s}_{i} ) + {\sum }_{μ = 1}^{M} {Γ}_{μ} ( {\sum }_{i = 1}^{N} {ω}_{i μ} ( {s}_{i} ) ) ]$$

where Z is a normalizing factor (the partition function) such that Σ_{S} P(S)=1, gᵢ(α) are column-specific amino acid fields, W_{iμ} is a sparse weight matrix for projecting the sequence into a continuous, M-dimensional space (termed the hidden unit space) and the potentials Γ_{μ}(I) are trainable, strictly convex non-linearities (such as quadratic functions).

Informally, the fields quantify amino acid preferences at each column. High scores are assigned to sequences if their amino acids match the preferred ones at each location. Each weight vector w×μ informally represents a sequence motif consistently found in a subset of the data. The projection I_{μ}(S)=Σᵢ w_{iμ}(sᵢ) quantifies the degree of matching between a given sequence and the motif, and the model allocates high probabilities to sequences that have either large positive or negative I_{μ}(S) via the quadratic-like non-linearity Γ_{μ}(I).

After training, novel sequences can be generated by combinatorial recomposition of positive and negative motif matches. The cRBM was shown to be a powerful inductive bias for protein sequence modeling, as it generalizes over single-site and pairwise Potts models by incorporating sparse, high-order epistatic interaction terms and is easier to interpret than a pairwise model or deep generative models. We trained multiple cRBM onto the multiple fragment alignment and selected one that maximized a trade-off between accuracy and interpretability (FIG. 8A, FIG. 8B).

To validate the model, its learnt log-probability function was compared to deep mutational scans of binding affinity recently performed by Nguyen et. al. (eLife. 2019 July; 8). The log-likelihood differences Δ log P were computed for all single-point mutants of four PxIxIT peptides: the natural fragments extracted from the human NFATc2 and AKAP79 proteins, and the synthetic PVIVIT and PKIVIT peptides (FIG. 3C, FIG. 3D). FIG. 3E shows a scatter plot between experimentally determined changes of binding affinity ΔΔG (lower is better) and predicted fitness change Δ log P (higher is better). An excellent correlation was found for the PKIVIT and PVIVIT peptides (Spearman correlation=p=0.70, 0.77 respectively), and a good one for NFATc2 and AKAP79 (−p=>0.52, 0.43 respectively). This agreement is substantially better than previous structure-based ΔΔG predictions performed using the Rosetta flex_ddG and FoldX ddG protocols (Nguyen et. al) (p=−0.2, 0.21 for PVIVIT and 0.21, 0.39 for AKAP79). It is also better than predicted fitness changes Δ long P computed using a PSSM (i.e. independent model) trained on the same alignment (Table 1).

TABLE 1
Predicting the impact of mutations on CaN binding affinity

| Dataset | Model | PKIVIT | PVIVIT | NFATc2 | AKAP79 |
|---|---|---|---|---|---|
| All mutations | cRBM | 0.76 | 0.77 | 0.52 | 0.43 |
| | PSSM | 0.74 | 0.72 | 0.39 | 0.13 |
| | Rosetta | NA | −0.2 | NA | 0.21 |
| | flex_ddG | | | | |
| | FoldX ddG | NA | 0.21 | NA | 0.39 |
| Flanking | cRBM | 0.52 | 0.5 | −0.12 | 0.3 |
| mutations | | | | | |
| | PSSM | 0.57 | 0.53 | −0.06 | −0.29 |
| | Rosetta | NA | −0.56 | NA | −0.09 |
| | flex_ddG | | | | |
| | FoldX ddG | NA | 0.31 | NA | 0.32 |

Table 1 compares mutation data and Rosetta/FoldX prediction from Nguyen et al, as above. The Spearman correlation coefficients between measured ΔΔ G and predicted changes (−Δ log P for cRBM and PSSM, ΔΔG for Rosetta/FoldX) are reported here.

While mutations of flanking residues were in general better tolerated than motif residues (FIG. 3C, FIG. 3D), the former was also found to be important as well. To quantify it, additional virtual deep mutational scans were performed for 100 representative natural fragments of the alignment (randomly sampled from the alignment by Kmeans++ algorithm), and the distribution of changes of likelihood upon mutation for each position (FIG. 8C) were calculated. Positions that least tolerated mutations on average were, in order, −1, P, I₁, +3, I₂ and −3. Additionally, visualization of the effective epistatic couplings between pairs of positions showed important covariation between central and flanking residues (especially −1). This showcases the importance of flanking residues for CaN binding.

Finally, it was investigated whether the model was able to identify common motifs shared between different substrates. To this end, the sequence motifs learnt were visualized (three representative motifs shown in FIG. 3F, together with their matching score distribution (FIG. 3G) and selected positive and negative top-matching fragments.

It was found that some motifs (shown in FIG. 3F), such as motif 1 (focusing on the central residues) and motif 3 (focusing on the C-terminal end) were gene-specific, as evidenced by the trimodal matching score distribution and the fact that its top-activating fragments came from identical substrates. Others, such as motif 2 (simultaneous presence of asparagine at position-1, an hydrophobic residue at position x₂ and negatively charged residues at positions +3 and +5), were found in fragments from various genes. The simultaneous presence or absence of negatively charged residues at location +3 and +5 was a recurring pattern. According to the present disclosure, it was hypothesized that they correspond to the simultaneous presence or absence of stabilizing salt bridges with the positively charged lysines/arginines of CaN at locations 100,318 and/or 332. Altogether, the sequence model recapitulated well-known biochemical features of the CaN interactions.

Next, the sequence model was used to generate two libraries of candidate peptides, respectively using regular Monte Carlo sampling and so-called low-temperature sampling to focus samples around with higher probability values, following Russ et al. (Science. 2020 Jul. 24; 369(6502):440-5). The former peptides spanned a larger portion of the sequence space and were on average further away from the set of natural sequences, while the latter had higher probability scores FIG. 8E). After redundancy reduction, 180 and 361 were selected for further analysis, respectively. Two known synthetic peptides (PVIVIT and PKIVIT), 74 representative natural binding fragments from the multiple fragment alignment, 36 purely random 16-length peptides, and 72 samples from the PSSM model were selected as controls.

FIGS. 3A-H show the generative modeling of PxIxIT binding motifs. FIG. 3A provides a schematic view of the generative approach. A “smooth” probability distribution over the whole sequence space is learnt from a limited number of samples. Unseen sequences with high probability are potential novel binders, whereas regions with low probability are likely non-functional proteins. FIG. 3B depicts the Restricted Boltzmann Machine, the parametric form chosen. FIGS. 3C and 3D provide the cRBM-predicted mutational landscapes for human NFATc2 and AKAP79 peptides. Red, white and blue entries correspond respectively to beneficial, neutral and deleterious mutations. FIG. 3E compares cRBM-predicted mutational landscapes with deep mutational scans of change in binding affinity measured by Nguyen et al. Four DMS were performed taking as wild type the PVIVIT, PKIVIT, NFATc2 and AKAP79 peptides. Spearman correlation coefficients are annotated. FIGS. 3F, 3G, and 3H show selected examples of sequence motifs learnt by the cRBM (FIG. 3F), together with their activity distribution (FIG. 3G) and top-activating sequences (FIG. 3H). Motif 1 is gene-specific, whereas motifs 2 and 3 are shared by multiple genes.

### Example 5: Library Refinement by Molecular Docking and Microarray Binding Assay (Step 3)

The sequence model a priori treats all natural sequences equally. However, their binding affinities span almost three orders of magnitude (0.5-250 uM). To further refine the list of candidate peptides, the docking energy score was estimated (where a lower score is better) using five available crystal structures of CaN bound to a PxIxIT-containing peptide and an ad-hoc template-based molecular docking followed by a flexible-backbone refinement pipeline based on Modeller and PepCrawler (FIG. 4A).

The docking energies were consistent from one CaN crystal structure to the other, and correlated with the likelihood of the SGM, Pearson correlation r=−0.21, p<10⁻⁸). Random or PSSM-designed sequences had significantly higher energy than natural or cRBM-designed ones (p<10⁻¹², two-sided Mann-Whitney-Wilcoxon test). In contrast, there was no statistically significant difference between natural fragments and cRBM designs. However, the distribution of energies for the random peptides (negative controls) and natural binding peptides (positive controls) overlapped significantly: 14% of random peptides had lower energy scores than at least half of the natural peptides (FIG. 4B). Hence, designing peptides solely based on docking would have likely led to a large number of false positives.

To rationalize the docking energy score from the peptide sequence, an additive single-site model was fitted to the docking results by sparse linear regression. The single-site model approximated the docking energies results (cross-validation Pearson correlation r=0.89). Visualization of the regression coefficients (FIG. 4C) confirmed that the motif sites were the most important ones, with highly hydrophobic side-chains (I,V,L,M,F) favored at these locations, as well as Proline at position “P”. Flanking residues were overall less important but also contributed, in particular at position +3, where negatively charged residues (D,E) were favored, consistently with the sequence model.

To evaluate the ability of the docking energy to discriminate between natural binders, approximate docking scores were predicted for all natural fragments using the single-site model and computed a per-substrate average (FIG. 4D). The substrate with the lowest average docking energy was the A-kinase anchoring protein 79 (AKAP79), which is consistent with previous binding affinity measurements (k_{d}˜0.1-2.0 uM) and its biological function. Indeed, AKAP79 tethers CaN to neural membranes next to synaptic clefts for phosphoregulation of synaptic signaling. NFAT lies in the middle of the spectrum, its affinity fine-tuned to an intermediate value that prevents over-activation of NFAT in the absence of calcium.

Altogether, it was concluded that the docking score can efficiently complement the evolutionary score by differentiating between natural genes with variable activation levels. On the other hand, peptide design based solely on the docking protocol would have resulted in a highly hydrophobic binding motif, presumably with low solvability and high reactivity, as well as limited accuracy for flanking residues.

In parallel, selected peptides were tested for CaN binding on a chip microarray (PEPperPRINT). 786 peptides were printed on the chip and were incubated with GST-tagged CaN overnight at 4° C. Following extensive washing, binding was detected by applying a fluorescently labeled (Alexa-Fluor 647) GST antibody. After additional washing and drying, the microarray slide was scanned (FIG. 4E), and fluorescent spots revealed peptides that bind CaN. The experiment was repeated five times, and Z-normalized fluorescence levels were determined following post-processing of the raw data (FIGS. 9A, 9B and 9C).

Although no statistically significant correlation was found between the experimentally determined fluorescence levels and either the sequence model or the docking scores, the positive outliers in the chip also had good sequence model and docking scores (FIG. 4F, FIG. 9D). In addition, a false negative readout was observed, as the PVIVIT and PKIVIT peptides had only weak fluorescence levels and fluorescence was not well explained a-posteriori by a sequence-based single-site model. It was concluded that the experimental fluorescence level alone did not consistently indicate binding, but that positive hits could be considered reliable. Based on the analysis of the sequence likelihood scores, docking scores and fluorescence levels on the chip, 15 peptides were selected for further analysis: the PVIVIT peptide, four native fragments and ten CRBM-designed peptides.

FIGS. 4A-F illustrate medium-throughput filtering by structural modeling and microarray screening. FIG. 4A depicts the structural modeling protocol: after alignment to the known PxIxIT binding site, an efficient flexible backbone structure refinement algorithm is applied to estimate the docking energy. FIG. 4B shows a histogram of docking energy scores for the generated peptides and selected controls (lower is better; normalized to zero mean and unit variance). FIG. 4C provides coefficients of the equivalent single-site model fitted by sparse linear regression, shown in weight logo representation. At each position, the height of the letter is proportional to the corresponding coefficient of the regression; residues with large negative coefficients (e.g. hydrophobic residues at the motif locations) contribute favorably to the docking score. Colors indicate physical property (black=hydrophobic, red=negatively charged, etc.). FIG. 4D provides the per-gene distribution of docking scores across natural fragments (lower is better). The docking protocol qualitatively discriminates between obligate and transient interactions. FIG. 4E provides an overview of the microarray screening. Peptides are printed on the chip (two circles per peptide). After pouring of CaN and subsequent washing, fluorescent-tagged, a CaN-targeting antibody is overlaid and an image is taken. Fluorescent spots indicate strong CaN binders. FIG. 4F shows a scatter plot of the sequence likelihood (normalized by length, higher is better) against fluorescence level (higher is better, see Methods for details of the data analysis).

### Example 6: In-Vitro Quantitative Binding Assay and Analysis (Step 4)

Selected peptides were synthesized and their ability to specifically bind the CaN PxIxIT binding site was evaluated by FP competition assay. Variable concentrations of each peptide were incubated in a solution of CaN complexed with fluorescently-labeled PVIVIT peptide, and FP levels indicating the peptide's ability to compete with the PVIVIT were read (FIG. 5). After fitting the polarization values to a single site inhibition model, the corresponding IC₅₀ values were extracted and reported in Table 2. It was found that 7/10 synthetic peptides and ¾ natural peptides successfully competed with PVIVIT for CaN binding, with IC₅₀ values ranging from 1.17 μM to 250 μM. For comparison, non-labeled PVIVIT were also tested for self-competition using an identical protocol, and an IC₅₀ of 10.2 μM was found.

FIG. 5 shows the FP competition assay of selected peptides for the binding of CaN to PVIVIT peptide. Variable concentrations of each selected peptide were incubated with CaN bound to the FITC-labeled PVIVIT peptide. Polarization levels were read and normalized values were fitted to a single site model. The curve shows the bound fraction of CaN to PVIVIT vs. the logarithmic concentration of the peptides.

Table 2 shows a list of natural and designed peptide sequences characterized by competitive FP assay. Abbreviations: Type: N-natural, C-control, D-designed; SEQ: SEQ ID NO; Nat.: closest natural peptide sequence; IC₅₀: half maximal inhibitory concentration in uM; #mut: number of mutations to closest natural sequence; Org (organism): HS: Homo sapiens; PeC: Pelecanus crispus; PrC: Propithecus coquereli; AL: Austrofundulus limnaeus; FG: Fulmarus glacialis; CC: Capronia coronata; SM: Schistosoma mansoni.

TABLE 2
properties of selected natural and designed
peptides

| Peptide | | | | | # | |
|---|---|---|---|---|---|---|
| Name | Type | SEQ | IC₅₀ | Sequence | mut | Org |
| C16Orf74 | N | 1 | 1.17 | KHLDVPDI | | HS |
| (Nat) | | | | IITPPTPT | | |
| PVIVIT | C | 4 | 10.2 | MAGPHPVI | | — |
| | | | | VITGPHEE | | |
| rbmTRESK | D | 5 | 14 | ADEAIPEI | | |
| | | | | VISKPEEP | | |
| Nat. | | 3 | — | ADEAVPQI | 6 | HS |
| TRESK | | | | IISAEELP | | |
| AKAP79 | N | 2 | 17.5 | KRMEPIAI | | HS |
| | | | | IITDTEIS | | |
| TRESK | N | 3 | 54 | ADEAVPQI | | HS |
| | | | | IISAEELP | | |
| rbmAKAP79 | D | 6 | 57 | AAGAGVGI | | |
| | | | | VITVTEAE | | |
| Nat. | | 11 | — | NAGAGVSI | 2 | PeC |
| AKAP79 | | | | VITVTEAE | | |
| rbmTRESK_ | D | 7 | 60 | ADEAIPEI | | |
| 2 | | | | TITSAELP | | |
| Nat. | | 12 | — | ADEAIPQI | 3 | PrC |
| TRESK | | | | TITAEELP | | |
| rbmAKAP79_ | D | 8 | 69 | ADGAGVGI | | |
| 2 | | | | VITVTEAE | | |
| Nat. | | 11 | — | NAGAGVSI | 2 | PeC |
| AKAP79 | | | | VITVTEAE | | |
| rbmRIPOR2 | D | 9 | 79 | ASVSNPEI | | |
| | | | | TVTSAETE | | |
| Nat. | | 13 | — | QSQSNPEI | 4 | AL |
| RIPOR2 | | | | TVTPPETE | | |
| rbmRIPOR2_ | D | 10 | 200 | HVSSSPRI | | |
| 2 | | | | TITPTQHR | | |
| Nat. | | 14 | — | HVSSSPDI | 2 | FG |
| RIPOR2 | | | | TATPTQHR | | |

The best peptide, a fragment of an open reading frame encoded by the human C16Orf74 gene selected for its high sequence score, had an IC_{50=1.17} uM. This was consistent with its high gene-averaged docking score (rank 5/67, FIG. 4D), and with recent binding affinity measurements for the CABIN1 gene, a close homolog. C16Orf74 features a highly hydrophobic PxIxIT-like motif, and, interestingly, a C-terminal proline-rich motif. Structural modeling with AlphaFold-multimer of C16Orf74 and PVIVIT suggests that the proline-rich peptide adopts a rigid polyproline helical conformation that effectively extends the beta sheet, enabling additional interaction surface with CaN without any entropic cost. It is important to note that such polyproline motif is extremely unlikely to be discovered by mutagenesis, as three simultaneous mutations to proline are required for the rigid structure to emerge. The second best natural peptide (IC_{50=17.5} uM) was a fragment of the human AKAP79 protein (SEQ ID NO: 2), in agreement with its high sequence score, gene-averaged docking score (rank 1/67, FIG. 4(D)) and previous studies.

The best synthetic peptide, ADEAIPEIVISKPEEP (SEQ ID NO: 5, rbmTRESK hereafter), was obtained by low-temperature sampling of the cRBM and bound CaN with comparable strength as PVIVIT (IC₅₀=14 uM). It featured six mutations from its closest natural counterpart, the CaN-binding fragment of human TRESK protein (SEQ ID NO: 3) and its IC₅₀ was almost four times lower (IC50=54 uM). A sequence with such a large number of mutations would have been difficult to reach via classical computational mutagenesis approach and almost impossible via experimental approach alone within a single screening round. Instead, rbmTRESK (human) was effectively obtained by rational recombination of the left flanking residues of Rattus NORVEGICUS TRESK (ADEAIPQIVIDAGADE, SEQ ID NO: 15), the motif residues of Salmo SALAR KCNN3 (PTQNPPEIVISSKEDS, SEQ ID NO: 16) and the right flanking residues of Ictidomys tridecemlineatus CAPN11 (TFWTNPQFKIYLPEED, SEQ ID NO: 17).

Interestingly, the above peptides all featured a PxIxIT-like motif, but this was not necessary: peptides rbmAKAP79 and rbmAKAP79_2, both similar to the AKAP79 protein of Pelicanus crispus, successfully competed with PVIVIT binding despite lacking proline residues.

Based on the sequences found, several consensus sequences were developed, further including permissible sequence variations which were predicted to maintain the binding to calcineurin. The consensus sequences are presented in Table 3.

TABLE 3
Consensus sequences

| | | SEQ |
|---|---|---|
| | | ID |
| Gene | Consensus sequence | No. |
| ALL | [A/T/S]X[P/V][E/K/Q/R/S/G]I | — |
| | [T/V/I][I/V][D/H/Q/S/T]XXE | |
| TRESK | AXP[E/K/Q/R/S]I[T/V/I][I/V] | 18 |
| | [D/H/Q/S/T]XXE | |
| AKAP79 | [A/T]GVGIVIT[I/P/V]TE | 19 |
| RIPOR2 | [A/S]NPEIT[I/V]TXAE | 20 |

### Example 7: In-Vitro Quantitative Binding Assays for Gene-Specific Peptides

Additional peptides were designed based on consensus sequences developed for each of the three genes TRESK, AKAP79, and RIPOR2. The newly designed peptides are presented in Table 4.

TABLE 4
newly designed peptides

| Gene | Peptide sequence | SEQ ID No. |
|---|---|---|
| TRESK | ADEANPEITITPPELP | 21 |
| | ADEAIPEITITPAELP | 22 |
| | ADEAIPKIVIHPPEEP | 23 |
| AKAP79 | NMGTGVGIVITITEAV | 24 |
| | PAGAGVGIVITVTEAE | 25 |
| RIPOR2 | ADEANPEITVTPAELP | 26 |
| | LSSSNPEITVTPAELD | 27 |
| | ASSANPEITVTPAELP | 28 |

The above peptides are synthesized and their ability to specifically bind the CaN PxIxIT binding site is evaluated by FP competition assay. Variable concentrations of each peptide are incubated in a solution of CaN complexed with fluorescently-labeled PVIVIT peptide, and FP levels indicating the peptide's ability to compete with the PVIVIT are read. After fitting the polarization values to a single site inhibition model, the corresponding IC₅₀ values are extracted. Based on results for similar peptides conforming to the consensus sequences indicated above, it is expected that the IC₅₀ values will be below 250 μM.

1.-28. (canceled)

29. A synthetic peptide capable of binding to calcineurin, wherein the synthetic peptide has a length of about 14-20 amino acids; has at least 1 amino acid difference from any natural peptide sequence; comprises a sequence conforming to a consensus sequence selected from the group consisting of SEQ ID NO: 18, SEQ ID NO: 19, and SEQ ID NO: 20; and binds calcineurin with an IC₅₀ of about 250 μM or less.

30. The synthetic peptide of claim 29, having about 1-6 amino acid differences from a natural peptide sequence that has the highest sequence identity with the synthetic peptide.

31. The synthetic peptide of claim 29, having a length of about 16 amino acids.

32. The synthetic peptide of claim 29, wherein the peptide sequence is most similar to a natural peptide sequence which is part of a protein selected from the group consisting of TRESK, AKAP79, and RIPOR2.

33. The synthetic peptide of claim 32, wherein the peptide sequence comprises a sequence conforming to a consensus sequences as set forth in SEQ ID NO: 18, and is most similar to a natural peptide sequence which is part of the TRESK protein.

34. The synthetic peptide of claim 32, wherein the peptide sequence comprises a sequence conforming to a consensus sequences as set forth in SEQ ID NO: 19, and is most similar to a natural peptide sequence which is part of the AKAP79 protein.

35. The synthetic peptide of claim 32, wherein the peptide sequence comprises a sequence conforming to a consensus sequences as set forth in SEQ ID NO: 20, and is most similar to a natural peptide sequence which is part of the RIPOR2 protein.

36. The synthetic peptide of claim 29, selected from the group consisting of SEQ ID Nos: 5-10 and 21-28.

37. The synthetic peptide of claim 29, wherein the binding is determined by competition with a PxIxIT motif-containing peptide.

38. The synthetic peptide of claim 37, wherein the PxIxIT motif-containing peptide has a sequence according to SEQ ID NO: 4.

39. A method of treating a subject in need of immunosuppression, comprising administering to the subject a therapeutically effective dose of the synthetic peptide of claim 29 or a pharmaceutical composition comprising it.

40. The method of claim 39, wherein the subject suffers from an autoimmune or an inflammatory disease or condition, or is a post-transplantation patient.

41. A computer-implemented method for designing protein-protein interaction modulator peptides, the method comprising the steps of:
identifying a binding region of a target protein;
identifying at least one substrate having a peptide-like binding fragment which interacts with the binding region of the target protein;
performing a homology/orthology search across sequence databases to identify additional homologous peptide-like binding fragments;
creating a data set comprising at least one peptide-like binding fragment and at least one homologous peptide-like binding fragment;
training a sequence generative model (GSM) to generate a library of candidate peptide sequences; and
screening the library of candidate peptide sequences for peptides capable of binding to the binding region of the target protein.

42. The computer-implemented method according to claim 41, wherein the screening comprises in-silico screening and/or in-vitro screening.

43. The computer-implemented method according to claim 42, wherein the in-silico screening comprises estimating the binding strength of at least one candidate peptide to the target protein by a protein-peptide docking algorithm.

44. The computer-implemented method according to claim 41, wherein the -silico screening comprises applying a template-based docking with Modeller followed by flexible backbone refinement with PepCrawler, or applying ab initio docking with AlphaFold-Multimer followed by ProteinMPNN for scoring.

45. The computer-implemented method according to claim 41, wherein the method further comprises the step of:
performing a quantitative binding assay on at least one candidate peptide to determine the ability of the at least one candidate peptide to compete with the binding of the at least one substrate.

46. The computer-implemented method according to claim 41, wherein the sequence generative model comprises a Boltzmann Machine and/or autoregressive model.

47. The computer-implemented method according to claim 46, wherein the Boltzmann Machine comprises a compositional Restricted Boltzmann Machine.

48. The computer-implemented method according to claim 41, wherein a two-stage sequence-based statistical filtering protocol is applied to results of the homology/orthology search to eliminate presumed non-interacting homologs.


SEQUENCE LISTING (28 sequences)

SEQ ID NO: 1 - protein, 16 aa, synthetic construct
KHLDVPDIIITPPTPT

SEQ ID NO: 2 - protein, 16 aa, synthetic construct
KRMEPIAIIITDTEIS

SEQ ID NO: 3 - protein, 16 aa, synthetic construct
ADEAVPQIIISAEELP

SEQ ID NO: 4 - protein, 16 aa, synthetic construct
MAGPHPVIVITGPHEE

SEQ ID NO: 5 - protein, 16 aa, synthetic construct
ADEAIPEIVISKPEEP

SEQ ID NO: 6 - protein, 16 aa, synthetic construct
AAGAGVGIVITVTEAE

SEQ ID NO: 7 - protein, 16 aa, synthetic construct
ADEAIPEITITSAELP

SEQ ID NO: 8 - protein, 16 aa, synthetic construct
ADGAGVGIVITVTEAE

SEQ ID NO: 9 - protein, 16 aa, synthetic construct
ASVSNPEITVTSAETE

SEQ ID NO: 10 - protein, 16 aa, synthetic construct
HVSSSPRITITPTQHR

SEQ ID NO: 11 - protein, 16 aa, synthetic construct
NAGAGVSIVITVTEAE

SEQ ID NO: 12 - protein, 16 aa, synthetic construct
ADEAIPQITITAEELP

SEQ ID NO: 13 - protein, 16 aa, synthetic construct
QSQSNPEITVTPPETE

SEQ ID NO: 14 - protein, 16 aa, synthetic construct
HVSSSPDITATPTQHR

SEQ ID NO: 15 - protein, 16 aa, synthetic construct
ADEAIPQIVIDAGADE

SEQ ID NO: 16 - protein, 16 aa, synthetic construct
PTQNPPEIVISSKEDS

SEQ ID NO: 17 - protein, 16 aa, synthetic construct
TFWTNPQFKIYLPEED

SEQ ID NO: 18 - protein, 11 aa, synthetic construct
  VARIANT 4: note = E or K or Q or R or S
  VARIANT 6: note = T or V or I
  VARIANT 7: note = I or V
  VARIANT 8: note = D or H or Q or S or T
AXPXIXXXXXE

SEQ ID NO: 19 - protein, 11 aa, synthetic construct
  VARIANT 1: note = A or T
  VARIANT 9: note = I or P or V
XGVGIVITXTE

SEQ ID NO: 20 - protein, 11 aa, synthetic construct
  VARIANT 1: note = A or S
  VARIANT 7: note = I or V
XNPEITXTXAE

SEQ ID NO: 21 - protein, 16 aa, synthetic construct
ADEANPEITITPPELP

SEQ ID NO: 22 - protein, 16 aa, synthetic construct
ADEAIPEITITPAELP

SEQ ID NO: 23 - protein, 16 aa, synthetic construct
ADEAIPKIVIHPPEEP

SEQ ID NO: 24 - protein, 16 aa, synthetic construct
NMGTGVGIVITITEAV

SEQ ID NO: 25 - protein, 16 aa, synthetic construct
PAGAGVGIVITVTEAE

SEQ ID NO: 26 - protein, 16 aa, synthetic construct
ADEANPEITVTPAELP

SEQ ID NO: 27 - protein, 16 aa, synthetic construct
LSSSNPEITVTPAELD

SEQ ID NO: 28 - protein, 16 aa, synthetic construct
ASSANPEITVTPAELP